(19) United States
(12) Patent Application Publication
(10) Pub. No.: US 2014/0213472 A1
(43) Pub. Date: Jul. 31, 2014

(54) Head and Neck Cancer Biomarkers

(71) Applicant: William Beaumont Hospital, Royal Oak, MI (US)

(72) Inventors: JAN A. AKERVALL, Ann Arbor, MI (US); Bryan J. Thibodeau, West Bloomfield, MI (US); George D. Wilson, West Bloomfield, MI (US)

(73) Assignee: William Beaumont Hospital, Royal Oak, MI (US)

(21) Appl. No.: 14/166,451

(22) Filed: Jan. 28, 2014

Related U.S. Application Data
(60) Provisional application No. 61/757,859, filed on Jan. 29, 2013.

(51) Int. Cl. C12Q 1/68; G01N 33/574

(52) U.S. Cl. CPC C12Q 1/6886; G01N 33/57407
USPC 506/9; 435/6.11; 435/7.1; 435/7.23; 435/7.4

(57) ABSTRACT

A method for predicting the response to treatment in a patient suffering from a head or neck cancer including: obtaining a biological sample for the patient; measuring the expression level of several gene and using the measurement to predict a patient's response to treatment. The methods may include predicting the treatment response in a patient having virally-induced head or neck cancer; and/or using the prediction to treat the patient.

This application claims priority to U.S. Ser. No. 61/757,859, filed Jan. 29, 2013. The entire contents of the aforementioned application are incorporated herein.

This application incorporates by reference in its entirety the Sequence Listing entitled “Biomarkers_Sequence_Listings_{—}332022_ST25(5).txt” (20.6 kilobytes), which was created on Jan. 28, 2014 and filed electronically herewith.

### BACKGROUND

Head and neck squamous cell carcinoma (HNSCC) has traditionally been associated with alcohol and tobacco use. Recently, a virally-induced form of HNSCC named “HPV-positive HNSCC” has become known. Some patients with HPV-positive HNSCC do not respond to treatment as well as other patients with HPV-positive HNSCC. As such, this new form of HNSCC may require different treatments for different patients, even though all suffer from HPV-positive HNSCC.

An infection by the human papillomavirus (HPV) is a primary cause of cervical cancer. An HNSCC cancer that is associated with an HPC infection is known as an “HPV-positive” HNSCC cancer, while an HNSCC cancer that is not believed to be associated with an HPC infection is known as an “HPV-negative” HNSCC cancer. Whereas HPV-negative HNSCC cancers overall have been on the decline (down 50% between 1984 and 2004), the incidence of HPV-positive HNSCC has increased by 225% over the same period. Today more than 70% of HNSCC of the oropharynx is HPV-positive, and the incidence is rising. Given the increasing incidence and improved response of HPV-positive head and neck patients, it is important to discern between those patients who will respond well and those who will not respond well to current treatment modalities. HPV-positive tumors are primarily located in the oropharynx, they are often non-keratinizing and poorly differentiated, and they are more likely to be present with local lymph node invasion. In spite of these undesirable phenotypes, HPV-positive tumors tend to demonstrate increased local control as well as better disease-free and overall survival compared to HPV-negative HNSCC patients. Tumors resulting from infection by a small group of high risk HPV subtypes, in particular HPV16, account for almost 90% of HPV-positive HNSCC.

The success of current therapies against HPV-positive HNSCC has introduced the idea of “therapy de-escalation.” Concomitant chemoradiation continues to be the mainstay of HNSCC treatment. Unfortunately, the treatment is associated with significant morbidity, despite development of chemo-IMRT and other radiation field and dose adjusting protocols, and in spite of protocols that provide for alternative chemotherapeutic agents with fewer side effects than traditional agents. Current standard of care does not depend on HPV status. If HNSCC HPV-positive patients truly are more responsive it would be possible to reduce the amount of chemotherapy, use radiation exclusively, or use other measures, in order to minimize treatment associated side effects.

Sometimes used as a surrogate marker for HPV infection, strong p16 expression has been shown to predict a favorable prognosis in HPV-positive HNSCC patients. Others have used morphologic changes to identify patient response. Spector et al. showed that patients with HPV-positive HNSCC and matted nodes were more likely to develop and die of distant metastasis. Spector M E, et. al. Matted and EGFR status. Head Neck 2012. A recent Radiation Therapy Oncology Group trial has been initiated to compare a radiation plus cetuximab arm to the current standard of care which is radiation plus cisplatin. See Mehra R. et. al., Semin Radiat Oncol 2012; 22(3):194-7.

Despite the fact that HPV-positive HNSCC patients show an improved response to therapy, there is a small minority of patients that do not respond. In order to optimize treatment and avoid unnecessary morbidity for these patients it would be highly beneficial to develop biomarkers to identify this sub-population. With the possible advancement of treatment de-escalation, it would also be important to identify patients that would respond positively before they started treatment.

Here, the inventors identify biomarkers and claim methods to differentiate between those HPV-positive HNSCC patients who will respond and those who will not respond well to current therapy standards, before treatment is initiated, thus providing assays and methods to assist clinicians in choosing optimal treatment regimens.

### BRIEF DESCRIPTION OF FIGURES

The following summary and detailed description of the invention will be better understood when read in conjunction with the figures. For the purpose of illustrating the invention, there are shown in the drawings, certain embodiment(s) which are presently preferred. It should be understood, however, that the invention is not limited to the precise arrangements and instrumentalities shown.

FIG. 1. Validation of microarray results utilizing RT-PCR. Values indicate fold-difference of Post-Tx Fails compared to CRs. Light gray: microarray result; dark gray: RT-PCR result.

FIG. 2. Hierarchical clustering based upon 49 genes that are differentially expressed between the Post-Tx Fails versus CRs (p≦0.01 and 1.5-fold cutoff). Patients are clustered on the horizontal axis (samples in orange correspond to the Post-Tx Fails; purple, CRs).

FIG. 3. Sub-network of genes regulated by E2F3, E2F4, and the E2F functional group. Pathway includes genes differentially expressed between Post-Tx Fails and CRs (ANOVA, p≦0.10 and fold-change≧1.2). Red is upregulated in Post-Tx Fails compared to CRs; blue, downregulated. P-value and fold-change requirement not met for E2F3 and E2F4.

### BRIEF DESCRIPTION OF SEQUENCE LISTINGS

We provide the nucleotide and amino acid sequences, respectively, for the six genes and proteins of interest in this application. See SEQ ID No. (s) 1-12. These sequences can also be found in the NCBI websites under names and numbers disclosed herein. The sequence listing provides one representative example of the gene or protein; other variants and mutations may be known and available, or made by one skilled in the art. The sequences provided in this application and the sequence listing is not intended and should not be used to limit the invention or claims unless expressly stated.

### SUMMARY OF THE INVENTION

This application describes and teaches methods for predicting the response to treatment, and for treating, a patient suffering from a head or neck tumor comprising: a) obtaining a biological sample of said tumor from said patient; b) measuring one or more of the protein, mRNA, or cDNA levels of at least one biomarker in said sample to obtain a biomarker measurement, wherein said biomarker is selected from the group consisting of: (a) LCE3D; (b) KRTDAP; (c) HMOX1; (d) KRT19; (e) MDK; and (f) TSPAN1; in any combination thereof; and using the measurement to predict a patient's response to treatment. The teachings herein can be used for head or neck tumors and where the head or neck tumor is a HNSCC and the HNSCC is a HPV-positive HNSCC. The named biomarkers may be useful where: a), b) and/or c), biomarkers, in any combination, show gene upregulation in said sample and, optionally, when any of biomarkers d), e) and/or f), in any combination, shows gene downregulation in said sample.

This application provides the methods wherein the protein, mRNA, or cDNA levels are determined by measuring the levels of one or more nucleic acid sequences, or fragments thereof, selected from the group consisting of a-f, as follows: (a) SEQ ID NO: 1 (LCE3D-cDNA); (b) SEQ ID NO: 2 (KRTDAP-cDNA); (c) SEQ ID NO: 3 (HMOX1-cDNA); (d) SEQ ID NO: 4 (KRT19-cDNA); (e) SEQ ID NO: 5 (MDK-cDNA); and (f) SEQ ID NO: 6 (cDNA-TSPAN1) and where the fragments are 15 or greater than about 15 consecutive nucleotides.

This application provides the methods wherein the protein, mRNA, or cDNA levels are determined by measuring the levels of one or more protein sequences, or fragments thereof, selected from the group consisting of a-f, as follows: (a) SEQ ID NO: 7 (LCE3D-Peptide); (b) SEQ ID NO: 8 (KRTDAP-Peptide); (c) SEQ ID NO: 9 (HMOX1-Peptide); (d) SEQ ID NO: 10 (KRT19-Peptide); (e) SEQ ID NO: 11 (MDK-Peptide); and (f) SEQ ID NO: 12 (TSPAN1-Peptide).

The method wherein said biomarker is selected from a gene selected from the group consisting of: (a) LCE3D; (b) KRTDAP; and (c) HMOX1; and using said measurement to predict a response for said patient, the prediction is based on whether the protein, mRNA, or cDNA levels of the measured biomarker(s) indicate biomarker levels are upregulated, or downregulated, or unchanged, is described. The method wherein the protein, mRNA, or cDNA levels of the measured biomarker(s) are upregulated, and said tumor is identified as HPV-positive HNSCC, then the prediction is that said patient will NOT respond well to traditional therapy is described. The method wherein when said protein, mRNA, or cDNA levels of the measured biomarker(s) are NOT upregulated and said tumor is identified as HPV-positive HNSCC, then the prediction is that said patient WILL respond well to traditional therapy is described.

The method wherein said biomarker is a gene selected from the group consisting of: (d) KRT19; (e) MDK; and (f) TSPAN1 and using said measurement to predict a response for said patient, where the prediction is based on whether the protein, mRNA, or cDNA levels of the measured biomarker(s) are upregulated, downregulated, or unchanged is described. The method wherein when said protein, mRNA, or cDNA levels of the measured biomarker(s) are observed to be DOWNregulated and the prediction is made that the head or neck tumor is one that is HPV-positive HNSCC and said patient will NOT respond well to traditional therapy. The method wherein when said protein, mRNA, or cDNA levels of the measured biomarker(s) are NOT observed to be DOWNregulated and said tumor is HPV-positive HNSCC, then the prediction is that the patient WILL respond well to traditional therapy is described.

The method of making a treatment recommendation for a patient suffering from a head or neck tumor, including wherein the head or neck tumor is a HNSCC and the HNSCC is a HPV-positive HNSCC, comprising: a) obtaining a biological sample of said tumor from said patient; b) measuring one or more of the protein, mRNA, or cDNA levels of at least one biomarker in said sample to obtain a biomarker measurement, wherein said biomarker is selected from the group consisting of (a) LCE3D; (b) KRTDAP; (c) HMOX1; (d) KRT19; (e) MDK; and (f) TSPAN1; in any combination thereof; and using said measurement to make a recommendation for treatment in said patient when any one or more of biomarkers (a-c), or any combination of biomarkers (a-c), indicate gene upregulation in said sample and/or any one or more of biomarkers (d-f), or any combination of biomarkers (d-f), indicate gene downregulation in said sample. Using the methods described here where the treatment is to provide the patient with non-traditional or alternate treatment modalities that do not include chemoradiation therapy.

Various combinations of the markers are claimed, both for predictive use and treatments, including all of the following options and combinations: wherein one of the following (a-j) groups of biomarkers are measured: a) only (1) biomarker is measured; b) at least two (2) biomarkers are measured; c) only two (2) biomarkers are measured; d) at least three (3) biomarkers are measured; e) only three (3) biomarkers are measured; 0 at least four (4) biomarkers are measured; g) only four (4) biomarkers are measured; h) at least five (5) biomarkers are measured; i) only five (5) biomarkers are measured; or j) (6) biomarkers are measured. We teach, describe and claim using the biomarkers LCE3D and KRTDAP that are measured. We teach, describe and claim using the biomarker wherein LCE3D is measured. We teach, describe and claim using the biomarker wherein KRTDAP is measured.

We teach, describe and claim a method for predicting the response to treatment, method of making a treatment recommendation, and the method of treatment involves a biological sample taken from a tumor, a cancerous tissue, a pre-cancerous tissue, a biopsy, blood, serum, saliva, or a tissue. We teach, describe and claim a method for predicting the response to treatment, method of making a treatment recommendation, and the method of treatment wherein said head or neck cancer is head and neck squamous cell carcinoma or HNSCC and/or said cancer is oropharyngeal or laryngeal squamous cell carcinoma and or said measurement is from a squamous cell carcinoma of the head and neck or HNSCC and a prediction is made to determine whether or not said patient will respond well to traditional therapy.

We teach, describe and claim a method for prognosis, or evaluation of the effectiveness of a treatment, of a solid tumor in a patient of interest, wherein said tumor is a HNSCC and said method of evaluation comprises obtaining a sample of said HNSCC, then determining whether said patient has a HPV-positive HNSCC, then further determining whether said patient will respond favorably to chemo or radiotherapy using the method for predicting the response to treatment, method of making a treatment recommendation, as described herein and where said HNSCC is determined to be HPV-positive HNSCC and where the patient is selected for “therapy de-escalation” including treatments that do not include chemoradiation.

We teach, describe and claim a kit for prognostic use, comprising: a panel of 1 to 6 biomarkers in any combination of any number from 1 to 6 comprising: at least one biomarker selected from the group consisting of a-f; wherein (a) LCE3D; (b) KRTDAP; (c) HMOX1; (d) KRT19; (e) MDK; and (f) TSPAN1.

We teach, describe and claim a method of determining a prognosis of cancer characterized by analysis of a sample comprising the following steps: a) determine if the sample is HPV-positive HNSCC or HPV-negative HNSCC; and if the sample is HPV-positive HNSCC then; b) determine whether the patient is likely or not likely to benefit from traditional therapy and wherein said determination is made based on whether or not at least one biomarker from the following group is obtained from the patient's tumor and where the biomarkers is said to be upregulated with biomarkers (a) LCE3D; (b) KRTDAP; (c) HMOX1; and downregulated in biomarkers selected from the group consisting of (d-f): (d) KRT19; (e) MDK; and (f) TSPAN1 and where said biomarkers may be in any number or combination. The determination of the protein levels may be carried out using immunohistochemistry, an immunoassay, a protein assay, mass spectrometry, immunofluorescence, or a combination thereof.

The preferred polynucleotide biomarkers have good reproducibility, and are relatively easy to extract and amplify using a chosen expression measurement protocol. In some embodiments of the invention, more than one polynucleotide biomarker may be used in a diagnostic test to give improved sensitivity, specificity, and positive and negative predictive value.

### DETAILED DESCRIPTION OF THE PREFERRED EMBODIMENTS

Before the subject invention is described further, it is to be understood that the invention is not limited to the particular embodiments of the invention described below, as variations of the particular embodiments may be made and still fall within the scope of the appended claims. The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter, which embodiments and Examples are provided by way of illustration, and are not intended to be limiting of the present invention, unless specified.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Although any methods, devices and materials similar or equivalent to those described herein can be used in the practice or testing of the invention, the preferred methods, devices and materials are now described.

### DEFINITIONS

“Amplification product” refers to an oligonucleotide sequence that whose copy number (i.e., concentration) has been increased using an amplification reaction, such as, for example, the polymerase chain reaction (PCR).

“Biological sample” is any fluid or other material derived from the body of a normal or diseased subject, such as tissue (e.g., brain tissue spinal tissue, liver tissue, etc.), blood, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. Methods of obtaining biological samples are well known in the art. Extraction of RNA, e.g., mRNA, from a biological sample such as blood plasma, cerebrospinal fluid, or brain tissue may be performed using well-known methods in the art.

“cDNA” refers to DNA oligonucleotide sequences that are completely or partially complementary; or completely or partially/substantially identical to RNA sequences (e.g., a polynucleotide sequence). As is well known the art, cDNA oligonucleotides may be generated directly from RNA oligonucleotides using the RNA sequence as a template, often by the use of a reverse transcriptase enzyme. cDNA may also be designed and directly synthesized using any sequencing method known in the art. The term “cDNA” includes sequences both (completely or partially) complementary to an RNA sequence and (completely or partially/substantially) identical to an RNA sequence (given the substitution of thymine (T) base for the uracil (U) present in the RNA sequence). cDNA sequences identical to the RNA sequence are often produced using a cDNA oligonucleotide with a complementary sequence as a template, for instance, using PCR, or any other synthetic method known in the art.

“Chemoradiation” means the same as chemoradiotherapy, which is a treatment that combines chemotherapy and radiotherapy, it is often used to treat advanced esophageal cancer.

“Chemotherapy” means the use of chemical agents in the treatment or control of disease.

“Differentially expressed”, “reduced levels” and “downregulated” or “elevated levels” and “upregulated” refer to the amount of expression or concentration of a polynucleotide or protein .in a biological sample from a patient suffering from a head or neck cancer, such as HPV-positive HNSCC, who has responded well to treatment compared to the amount of the polynucleotide or protein in a biological sample from a control. The control may be patients suffering from a head or neck cancer, such as HPV-positive HNSCC, who has not responded well to treatment; or individual(s) that do not have HNSCC, have HNSCC (or a particular severity or stage of HNSCC), or have other reference diseases. For example, a polynucleotide that is downregulated in a biological sample from HNSCC patients is present at lower concentration in biological sample from HNSCC patients than in a biological sample from a subject who does not have HNSCC. For certain polynucleotides, elevated levels in a biological sample indicates the presence of or a risk for HNSCC; at the same time, other polynucleotides may be present in reduced levels in patients or subjects with HNSCC. In either of these example situations, polynucleotides are “differentially expressed” in HNSCC subjects and healthy controls.

“DNA coding sequence” or a “nucleotide sequence encoding” a particular protein is a DNA sequence that is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. “Non-coding” genomic sequences may include regulatory, RNA transcription sequences (rRNA, tRNA, polynucleotide, etc.), introns and other non-gene sequences, such as structural sequences, putatively non-functional sequences (“junk DNA”) and the like.

“HMOX1” is listed in the NCBI data base as “Homo sapiens heme oxygenase (decycling) 1 (HMOX1)”, with the reference number for the mRNA being (NM_{—}002133.2) and for the gene (NG_{—}023030.1.) The protein reference number is (NP_{—}002124.1). Known as heme oxygenase 1, it may also be known as heat shock protein, 32-kD, it is also known as bK286B110; HO-1 and HSP32. The mRNA or cDNA and peptide sequences to this gene and protein are provided in the sequence listing as SEQ. ID. NO.s 3 and 9, respectively. Heme oxygenase occurs as 2 isozymes, an inducible heme oxygenase-1 and a constitutive heme oxygenase-2. HMOX1 and HMOX2 belong to the heme oxygenase family.

“HNSCC” is head and neck squamous cell carcinoma.

“HPV” is the human papillomavirus (HPV) infection or virus.

“Hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized,” and may form stem and loop structures or the like under certain conditions.

“Isolated nucleic acid molecule” is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

“Isolated polynucleotide” or “Isolated polypeptide” is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

“KRT19” or “keratin 19” or “NP_{—}002267” is named by NCBI as “Homo sapiens keratin 19” or (KRT19), mRNA reference number is (NM_{—}002276.4). Synonyms are CK19, K19, K1 CS, 40-kD, 40-kDa keratin intermediate filament, CK-19, cytoderatin 19; keratin, keratin-19, type 1 cytoskeletal 19; The mRNA or cDNA and peptide sequences to this gene and protein are provided in the sequence listing as SEQ. ID. NO.s 4 and 10, respectively.

“KRTDAP” or keratinocyte differentiation-associated protein isoform 1 precursor has NCBI accession or reference number (NM_{—}207392.2) for the mRNA, (NG_{—}030050.1) for the gene and (NP_{—}997275.1) for the protein. The mRNA or cDNA and peptide sequences to this gene and protein are provided in the sequence listing as SEQ. ID. NO.s 2 and 8, respectively.

“LCE3D” has NCBI accession reference number (NM_{—}032563.1) for its mRNA or cDNA and (NP_{—}115952.1) for its protein. The gene and/or protein may be referred to by other names or numbers such as “late cornified envelope 3D (LCE3D)” or (LCE3D gene) and is also known as (LEP16), (SPRL6A) and (SPRL6B). The cDNA or mRNA and peptide sequences to this gene and protein are provided in the sequence listing as SEQ. ID. NO.s 1 and 7, respectively.

“MDK” or “midkine” is Homo sapiens midkine (neurite growth-promoting factor 2) (MDK), transcript variant 1, and the mRNA cDNA has NCBI Reference Sequence No.: (NM_{—}001012334.2) and the protein (NP_{—}00102334.1). The mRNA or cDNA and peptide sequences to this gene and protein are provided in the sequence listing as SEQ. ID. NO.s 5 and 11, respectively.

“NCBI” is National Center for Biotechnology Information, the US government website is www.ncbi.nim.nih.gov. The genes and proteins mentioned here, and especially the following LCE3D, KRTDAP, HMOX1, KRT19, MDK and TSPAN1 can be found in NCBI reference sequence collection, reference or accession numbers, and are provided herein.

“Nucleic acids” encompass nucleotides of RNA and DNA, including cDNA (DNA transcribed from RNA template strands), genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using nucleotide analogs or derivatives (e.g., inosine or phosphorothioate nucleotides). Such oligonucleotides can be used, for example, to prepare nucleic acids that have altered base-pairing abilities or increased resistance to nucleases.

“Percent identity” can be determined by hybridization of polynucleotides under conditions that form stable duplexes between similar regions, followed by digestion with single-stranded-specific nuclease(s), and size determination of the digested fragments. DNA sequences that are homologous can be identified in a Southern hybridization experiment under, for example, stringent conditions, as defined for that particular system. Defining appropriate hybridization conditions is within the skill of the art. See, e.g., Sambrook et al. supra; DNA Cloning, supra; Nucleic Acid Hybridization, supra.

“Physiological conditions” refers to specific stringency conditions that approximate or are conditions inside an animal (e.g., a human). Exemplary physiological conditions for use in vitro include, but are not limited to, 37° C., 95% air, 5% CO2, commercial medium for culture of mammalian cells (e.g., DMEM media available from Gibco, MD), 5-10% serum (e.g., calf serum or horse serum), additional buffers, and optionally hormone (e.g., insulin and epidermal growth factor).

“Polynucleotide” is a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

“Polynucleotide encoding a protein” as used herein refers to a DNA fragment or isolated DNA molecule encoding a protein, or the complementary strand thereto; but, RNA is not excluded, as it is understood in the art that thymidine (T) in a DNA sequence is considered equal to uracil (U) in an RNA sequence. Thus, RNA sequences for use in the invention, e.g., for use in RNA vectors, can be derived from DNA sequences, by thymidine (T) in the DNA

“Primer” means an oligonucleotide sequence that is capable of initiating or facilitating transcription or translation of a template oligonucleotide by binding or hybridizing to a template or target oligonucleotide. In some instances, a primer may contain one or more sequences that are complementary to the template or target oligonucleotide.

“Probe” is used to detect the presence of an oligonucleotide in a sample, often by selectively binding or hybridizing to all or portion of the oligonucleotide sequence. In some aspects, probes may be oligonucleotides, antibodies, and the like. Probes may be “tagged” with a detection label to aid in observation or detection of the oligonucleotide. “Tagging” may include covalent, ionic, hydrogen or other chemical bonding to a fluorophore, radiolabelling a portion or one or more atoms of the probe, and the like. Examples of probes and tagging techniques are provided more fully below.

“Radiotherapy” means the treatment of disease by means of radiation such as with X-rays, and is also called radiation therapy.

“Region”, when in reference to an oligonucleotide (as in “a region of a given oligonucleotide”) refers to a contiguous portion of the entire oligonucleotide sequence. Regions may range in size from 3-4 bases to the entire sequence minus one base.

“Sequence identity” is a percent identity between two polynucleotide or two polypeptide moieties. Genes that share a high sequence identity or similarity support the hypothesis that they share a common ancestor and are therefore homologous. Sequence homology may also indicate common function. Two DNA, or two polypeptide sequences are similar to each other and may be homologous when the sequences exhibit at least about 80%, 81%, 82%, 83%, 84%, 85%, 86%, 87%, 88%, 89%, preferably at least about 90%, 91%, 92%, 93%, 94% and most preferably at least about 95%, 96%, 97%, 98%, 99%, 99.5%, 99.9% sequence identity over a defined length of the molecules. As used herein, sequence identity also refers to sequences showing complete identity (100% sequence identity) to the specified DNA or polypeptide sequence.

“Subject” or “Patient” as used herein refers to a mammal, preferably a human, in need of diagnosis and/or treatment for a condition, disorder or disease.

“Substantially homologous”, when used in reference to a double-stranded nucleic acid sequence such as a cDNA or genomic clone, it refers to any probe that can hybridize to either or both strands of the double-stranded nucleic acid sequence under conditions of low stringency. “TSPAN1” or “tetraspanin 1” is Homo sapiens tetraspanin 1 (TSPAN1). The mRNANCBI Reference Sequence is (NM_{—}005727.3), gene synonyms are “NET1”, “TM4C” and “TM4SF”; and the protein has NCBI Reference Sequence: (NM_{—}005718.2). The mRNA or cDNA and peptide sequences to this gene and protein are provided in the sequence listing as SEQ. ID. NO.s 6 and 12, respectively.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well-known and commonly employed in the art. Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

### DETAILED DESCRIPTION

Specific genes as well as the cell cycle cell process of patients with HPV-positive HNSCC are identified so that responders and non-responders in advance of traditional therapy, such as chemoradiation treatments, can be identified. Shown herein are results of studies that permit identification of identify patients who will become either Complete Responders (CRs) or Post-Treatment Failures (Post-TS Fails) as measured by the patient's later response to chemoradiation therapy.

In one aspect, the present invention provides for methods for predicting a response to chemoradiotherapy treatment in a patient suffering from a head or neck cancer such as HPV-positive HNSCC. In some embodiments, the discovery here involves head or neck cancer such as squamous cell carcinoma of the head and neck. In other embodiments, the cancer is oropharyngeal or laryngeal squamous cell carcinoma. Therapy of these tumors can also include drug therapy, chemotherapy, hormone therapy, radiotherapy, immunotherapy, surgery, gene therapy, anti-angiogenesis therapy, palliative therapy, or other conventional or experimental therapies or a combination thereof, in addition to treatment with chemoradiation.

In some embodiments the discovery disclosed herein involves obtaining a biological sample from a patient and measuring the protein, or mRNA levels of one or more of the following markers: late cornified envelope 3D (LCE3D); keratinocyte differentiation-associated protein (KRTDAP); heme oxygenase 1 (HMOX1); tetraspanin 1 (TSPAN1); midkine (MDK also known as neurite growth-promoting factor 2); and keratin 19 (KRT19). The discovery may involve and use measurements of just one, two, three, four, five or all six and or any combination of these 6 markers.

Some of these markers are often highly upregulated in HPV-positive HNSCC. Frequently upregulated genes are: late cornified envelope 3D (LCE3D), keratinocyte differentiation-associated protein (KRTDAP), and heme oxygenase 1 (HMOX1). Some of the markers are more frequently downregulated and they include: tetraspanin 1 (TSPAN1), midkine (MDK, also known as neurite growth-promoting factor 2), and keratin 19 (KRT19).

In some embodiments the methods here involve measuring whether or not any combination of one, two or three of LCE3D, KRTDAP, and HMOX1 are upregulated and either separately or in combination with the measurement of LCE3D, KRTDAP, and HMOX1 whether or not any combination of one, two or three of TSPAN1, MDK, and KRT19 are downregulated. The disclosure and claims here may involve and use measurements of just one, two, three, four, five or all six and or any combination of 1-6 of these markers, either as up or down regulated.

The cDNA/mRNA sequences and amino acid sequences for each protein are: late cornified envelope 3D (LCE3D) is SEQ. ID. NO: 1.+7; keratinocyte differentiation-associated protein (KRTDAP) is SEQ. ID. NO: 2+8; heme oxygenase 1 (HMOX1) is SEQ. ID. NO: 3+9; tetraspanin 1 (TSPAN1) is SEQ. ID. NO: 4+10; midkine (MDK also known as neurite growth-promoting factor 2) is SEQ. ID. NO: 5+11; and keratin 19 (KRT19) is SEQ. ID. NO: 6+12. These sequences should be considered as non-limiting examples only. Other variants of these proteins may exist and become known to those skilled in the art.

Very strong upregulation of cornified envelope 3D (LCE3D) and keratinocyte differentiation-associated protein (KRTDAP) were found in the Post-Tx Fails compared to the CRs. Both LCE3D and KRTDAP are associated with keratinocytes and keratinization in the skin. LCE3D is known to be associated with keratinization in the skin, it is also associated with epidermal differentiation and expressed in both esophagus and tongue. KRTDAP has a role in the regulation of keratinocyte differentiation and maintenance of stratified epithelia and is highly expressed in oral mucosa and tongue.

Another gene elevated in Post-Tx Fails is heme oxygenase 1 (HMOX1). There are contradictory results in regards to the association of HMOX1 and lymph node metastasis. One study of SCC of the tongue associated low HMOX1 with metastasis (Yanagawa T et. al. Oral Oncol 2004; 40(1):21-7) while another study of areca-quid-chewing associated high HMOX1 expression with lymph node positivity (Lee S S, et. al. J Formos Med. Assoc. 2008; 107(5):355-63). Other studies have demonstrated an association between HMOX1 and treatment effectiveness. The role of HMOX1 in cisplatin treatment of colon cancer xenografts was examined and it was found that the inhibition of HMOX1 expression increased the efficacy of cisplatin treatment. See, Lee C K et. al. Food Chem Toxicol 2012; 50(7); 2565-74.

The most downregulated genes in patients with HPV-positive HNSCC who fail to respond to chemoradiation we found to be: tetraspanin 1 (TSPAN1), midkine (MDK, also known as neurite growth-promoting factor 2), and keratin 19 (KRT19).

We note that KRT19 has been identified as a variably expressed marker in oral SCC that was under expressed in well-differentiated compared to poorly differentiated oral SCC²⁶ (Khanom R et al. Histol Histopathol 2012; 27(7): 949-59) MDK encodes a secreted growth factor that binds heparin that acts as a multifunctional cytokine²⁷ (Fujita S. et al. Hum Pathol 2008; 39(5):694-700) Interestingly the preponderance of evidence shows that increased MDK expression is less favorable in oral SCC and associated with tumor progression²⁸ (Fujita S. et al. Hum Pathol 2008; 39(5):694-700) (Jham B C et al. J Oral Pathol Med 2012; 41(1): 21-6) and decreased 5-year survival²⁹ (Ota K, Fujimori H et al. Int J Oncol 2010; 37(4): 797-804). However, a recent study by Ota et al. exhibited that downregulation of MDK (5) resulted in decreased sensitivity of oral SCC to cisplatin³⁰ (Ota T. Jono H et al. Oncol Rep 2012; 27 (5): 1674-80).

In addition to our disclosure of specific gene expression changes, a broad-spectrum of differences between Post-Tx Fails and CRs were identified. Differences between the patient populations were found in all of the following: in cell processes of genome stability, in cell cycle, and in DNA repair (Table 1).

TABLE 1
The Patient and Lesion Characteristics for the Samples

| Patient ID | Response | Sex | Age (dx) | PRIM SITE | STAGE | TNM | Status | Tobacco | Alcohol |
|---|---|---|---|---|---|---|---|---|---|
| D383 | CR | m | 60 | epiglottis | 3 | T3N0M0 | NED | prev (40) | prev (heavy) |
| D226 | CR | m | 79 | supraglottis | 2 | T2N0M0 | DOD | yes (60) | prev (heavy) |
| D408 | CR | f | 85 | vocal cord; right | 3 | T3N0M0 | NED | prev | yes (social) |
| E25 | CR | m | 71 | tonsil, left | 4a | T3N2aM0 | NED | no | no |
| G385 | CR | m | 81 | tonsil, right | 3 | T2N1M0 | NED | prev (40) | yes (social) |
| I306 | CR | f | 69 | tonsil, left | 2 | T2N0M0 | NED | no | no |
| J315 | CR | m | 62 | bot | 4a | T1N2cM0 | NED | no | no |
| J272 | CR | f | 64 | tonsil; right | 4a | T2N2bM0 | NED | no | yes (social) |
| J381 | CR | f | 65 | tonsil; left | 3 | T2N1M0 | NED | no | no |
| K388 | CR | m | 50 | true vocal cord; | 2 | T2N0M0 | NED | yes (31) | yes (1 daily) |
| | | | | left | | | | | |
| R1138 | CR | m | 51 | bot | 3 | T2N1M0 | NED | prev (10) | yes (0.5 daily) |
| T387 | CR | m | 46 | tonsil | 4a | T2N2M0 | ? | no | yes (social) |
| B307 | Post-Tx | m | 61 | tonsil | 4a | T3N2cM0 | DOD | yes (100) | yes (heavy) |
| | Fail | | | | | | | | |
| B242 | Post-Tx | f | 87 | post pharyngeal | 2 | T2N0M0 | DOD | prev (10) | no |
| | Fail | | | wall | | | | | |
| D325 | Post-Tx | m | 72 | tongue, right | 4a | T4N2bM0 | DOD | prev (10) | prev (2 daily) |
| | Fail | | | | | | | | |
| D665 | Post-Tx | m | 67 | left tongue base | 3 | T3N0M0 | NED | prev (30) | no |
| | Fail | | | | | | | | |
| D1302 | Post-Tx | m | 57 | R maxillary & | 4a | T2N2bM0 | NED | yes (60) | yes (social) |
| | Fail | | | b/l pyriform | | | | | |
| G7 | Post-Tx | m | 64 | recurrent tongue, | N/A | T2N0M0 | DOD | no | no |
| | Fail | | | left | | | | | |
| G27 | Post-Tx | m | 56 | FOM extending to | N/A | N/A | DOD | prev (100) | prev (heavy) |
| | Fail | | | tongue | | | | | |

Clinical Data. Response: Complete Responder (CR) and Post-treatment Failure (Post-Tx Fail). Status: No evidence of disease (NED) or dead of disease (DOD). Tobacco Use: previous use (prev) current use (yes), and estimated pack years if known. Alcohol use: previous use (prev), current use (yes), and self-reported amount if known.

Table 2 shows sub-networks of genes/proteins regulating cell processes that are highly represented by genes that are differentially expressed between the Post-Tx Fails and CRs (≦0.05 and 1.5-fold cutoff)

TABLE 2
Highly Regulated Cell Processes

| Proteins Regulating the Cell | |
|---|---|
| Processes of: | p-value |
| genome stability | 1.58E−09 |
| genetic instability | 1.06E−07 |
| genome instability | 1.66E−07 |
| mitosis | 1.73E−07 |
| cell cycle checkpoint | 5.25E−07 |
| cell cycle | 8.83E−07 |
| S phase | 1.35E−06 |
| response to DNA damage | 2.69E−06 |
| meiosis | 2.71E−06 |
| DNA repair | 8.45E−06 |
| chromosome segregation | 9.70E−06 |
| single-stranded DNA binding | 1.39E−05 |
| DNA strand elongation | 1.94E−05 |
| kinetochore assembly | 2.46E−05 |
| cell proliferation | 3.91E−05 |

Table 2: the top 15 cellular processes that are highly regulated between the CRs and Post-Tx Fails. These highly regulated categories consist of two main groups: DNA damage-related (genome stability, genetic instability, genome instability, response to DNA damage, and DNA repair) and cell cycle-related (mitosis, cell cycle checkpoint, cell cycle, S phase, cell proliferation).

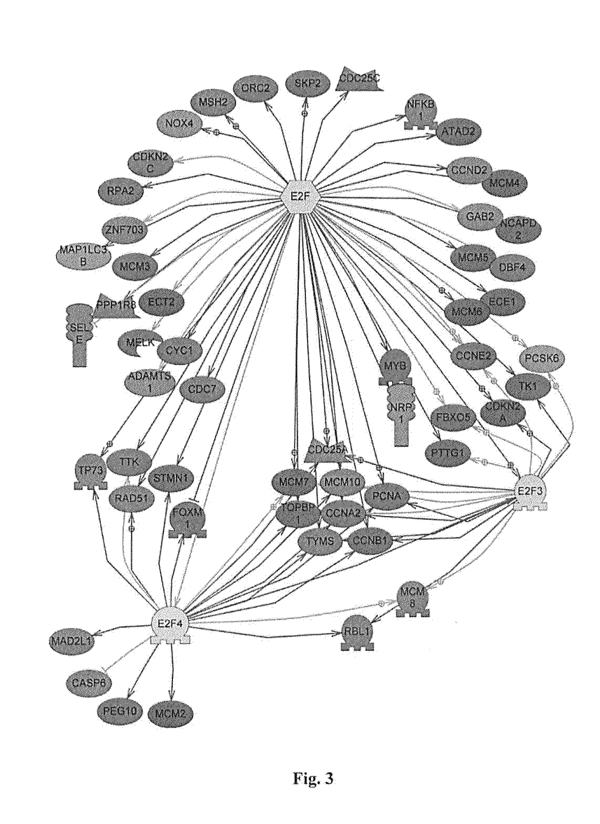

Utilizing analysis that does not include any predefined cutoffs it also was determined that cell cycle was highly regulated between the Post-Tx Fails and CRs. Sub-network enrichment analysis (SNEA) found sub-networks centered on genes associated with E2F, in particular E2F3 and E2F4, as highly regulated. E2F3 protein binds specifically to retinoblastoma protein pRB in a cell cycle-dependent manner while E2F4 protein binds to the three tumor suppressor proteins pRB, p107 and p130. See Table 3. Closer examination of the sub-network (FIG. 3) reveals several groups of cell cycle-associated genes: cyclins (CCNA2, CCNB1, CCND2, CCNE2), cyclin-dependent kinases (CDKN2A, CDKN2C), and mini-chromosome maintenance proteins that are involved in the initiation of eukaryotic genome replication (MCM2, MCM3, MCM4, MCM5, MCM6, MCM7, MCM8, MCM10). The vast majority of these cell-cycle associated genes are downregulated in the Post-Tx Fail patients. These combined results suggest that alterations in the cell cycle characteristics of the tumor may result in the different responses seen following treatment. We found Post-Tx Fails have a reduced or slowed capacity for cell cycle progression compared to the CRs.

Table 3 (a) (b) (c)

TABLE 3 (a)
Genome Stability/Genetic Instability/Genome Instability

| | | P-t Fail vs. |
|---|---|---|
| Gene Symbol | Gene Name | CR |
| ARHGAP1 | Rho GTPase activating protein 1 | 1.50 |
| ATMIN | ATM interactor | 1.53 |
| BARD1 | BRCA1 associated RING domain 1 | −1.60 |
| CCNB1 | cyclin B1 | −2.05 |
| FANCD2 | Fanconi anemia, complementation group D2 | −1.75 |
| FBXO5 | F-box protein 5 | −1.73 |
| FEN1 | flap structure-specific endonuclease 1 | −1.67 |
| H2AFZ | H2A histone family, member Z | −1.78 |
| HIF1A | hypoxia inducible factor 1, alpha subunit | 1.82 |
| HMOX1 | heme oxygenase (decycling) 1 | 2.99 |
| MCM2 | minichromosome maintenance complex component 2 | −2.65 |
| MCM3 | minichromosome maintenance complex component 3 | −1.74 |
| MCM4 | minichromosome maintenance complex component 4 | −2.06 |
| MCM6 | minichromosome maintenance complex component 6 | −2.02 |
| MECOM | MDS1 and EVI1 complex locus | −2.08 |
| MSH6 | mutS homolog 6 (E. coli) | −1.68 |
| MYB | v-myb myeloblastosis viral oncogene homolog (avian) | −1.71 |
| PARP1 | poly (ADP-ribose) polymerase 1 | −1.57 |
| PCNA | proliferating cell nuclear antigen | −1.72 |
| PTEN | phosphatase and tensin homolog | 1.95 |
| PTTG1 | pituitary tumor-transforming 1 | −2.08 |
| RAD51 | RAD51 homolog (RecA homolog, E. coli) (S. cerevisiae) | −1.68 |
| RAD51AP1 | RAD51 associated protein 1 | −1.78 |
| RAD54B | RAD54 homolog B (S. cerevisiae) | −1.58 |
| SGOL1 | shugoshin-like 1 (S. pombe) | −1.96 |
| TTK | TTK protein kinase | −1.79 |

TABLE 3 (b)
Mitosis/Cell Cycle

| | | P-t Fail vs. |
|---|---|---|
| Gene Symbol | Gene Name | CR |
| AGR2 | anterior gradient homolog 2 (Xenopus laevis) | −2.23 |
| ARRB1 | arrestin, beta 1 | 2.63 |
| ATMIN | ATM interactor | 1.53 |
| BARD1 | BRCA1 associated RING domain 1 | −1.60 |
| CASP9 | caspase 9, apoptosis-related cysteine peptidase | −1.58 |
| CCNB1 | cyclin B1 | −2.05 |
| CENPF | centromere protein F, 350/400ka (mitosin) | −2.10 |
| CYP24A1 | cytochrome P450, family 24, subfamily A, | −4.19 |
| | polypeptide 1 | |
| DCUN1D3 | DCN1, defective in cullin neddylation 1, domain | 2.02 |
| | containing 3 (S. cerevisiae) | |
| DTL | denticleless homolog (Drosophila) | −2.75 |
| EFHD2 | EF-hand domain family, member D2 | −1.70 |
| FANCD2 | Fanconi anemia, complementation group D2 | −1.75 |
| FBXO5 | F-box protein 5 | −1.73 |
| FEN1 | flap structure-specific endonuclease 1 | −1.67 |
| GAB2 | GRB2-associated binding protein 2 | 2.00 |
| H2AFZ | H2A histone family, member Z | −1.78 |
| HES1 | hairy and enhancer of split 1, (Drosophila) | −1.65 |
| HIF1A | hypoxia inducible factor 1, alpha subunit (basic helix- | 1.82 |
| | loop-helix transcription factor) | |
| HJURP | Holliday junction recognition protein | −1.58 |
| HMOX1 | heme oxygenase (decycling) 1 | 2.99 |
| ING4 | inhibitor of growth family, member 4 | −1.51 |
| ITGA2 | integrin, alpha 2 (CD49B, alpha 2 subunit of VLA-2 | −2.26 |
| | receptor) | |
| KRT18 | keratin 18 | −1.71 |
| KRT19 | keratin 19 | −3.13 |
| LAPTM4B | lysosomal protein transmembrane 4 beta | −2.01 |
| LIN9 | lin-9 homolog (C. elegans) | −1.54 |
| LMNB1 | lamin B1 | −1.89 |
| LRIG1 | leucine-rich repeats and immunoglobulin-like | −1.83 |
| | domains 1 | |
| MAP3K3 | mitogen-activated protein kinase kinase kinase 3 | 1.53 |
| MCM2 | minichromosome maintenance complex component 2 | −2.65 |
| MCM3 | minichromosome maintenance complex component 3 | −1.74 |
| MCM4 | minichromosome maintenance complex component 4 | −2.06 |
| MCM6 | minichromosome maintenance complex component 6 | −2.02 |
| MDK | midkine (neurite growth-promoting factor 2) | −3.47 |
| MECOM | MDS1 and EVI1 complex locus | −2.08 |
| METAP1 | methionyl aminopeptidase 1 | −1.60 |
| MLF1 | myeloid leukemia factor 1 | −1.92 |
| MSH6 | mutS homolog 6 (E. coli) | −1.68 |
| MSR1 | macrophage scavenger receptor 1 | 2.66 |
| MYB | v-myb myeloblastosis viral oncogene homolog | −1.71 |
| | (avian) | |
| MYO5A | myosin VA (heavy chain 12, myoxin) | 2.10 |
| NCAPH | non-SMC condensin I complex, subunit H | −1.79 |
| NDEL1 | nudE nuclear distribution gene E homolog (A. nidulans)- | 1.59 |
| | like 1 | |
| NEK2 | NIMA (never in mitosis gene a)-related kinase 2 | −1.87 |
| NOX4 | NADPH oxidase 4 | 1.59 |
| NUF2 | NUF2, NDC80 kinetochore complex component, | −1.69 |
| | homolog (S. cerevisiae) | |
| NUP155 | nucleoporin 155 kDa | −1.62 |
| NUP210 | nucleoporin 210 kDa | −2.22 |
| OIP5 | Opa interacting protein 5 | −1.68 |
| PARP1 | poly (ADP-ribose) polymerase 1 | −1.57 |
| PCNA | proliferating cell nuclear antigen | −1.72 |
| PEG10 | paternally expressed 10 | −2.13 |
| PER1 | period homolog 1 (Drosophila) | 1.62 |
| PKIA | protein kinase (cAMP-dependent, catalytic) inhibitor | −1.98 |
| | alpha | |
| PLD2 | phospholipase D2 | 1.67 |
| PTEN | phosphatase and tensin homolog | 1.95 |
| PTTG1 | pituitary tumor-transforming 1 | −2.08 |
| RAD51 | RAD51 homolog (RecA homolog, E. coli) (S. cerevisiae) | −1.68 |
| RAD51AP1 | RAD51 associated protein 1 | −1.78 |
| RAD54B | RAD54 homolog B (S. cerevisiae) | −1.58 |
| RASSF3 | Ras association (RalGDS/AF-6) domain family | 1.71 |
| | member 3 | |
| RBBP8 | retinoblastoma binding protein 8 | −1.69 |
| RPA2 | replication protein A2, 32 kDa | −1.65 |
| SESN3 | sestrin 3 | 2.15 |
| SGOL1 | shugoshin-like 1 (S. pombe) | −1.96 |
| SMC2 | structural maintenance of chromosomes 2 | −1.80 |
| SSBP2 | single-stranded DNA binding protein 2 | −1.77 |
| STMN1 | stathmin 1 | −1.81 |
| TFDP2 | transcription factor Dp-2 (E2F dimerization partner 2) | −1.94 |
| TK1 | thymidine kinase 1, soluble | −1.82 |
| TPPP3 | tubulin polymerization-promoting protein family | −3.49 |
| | member 3 | |
| TTK | TTK protein kinase | −1.79 |
| UHMK1 | U2AF homology motif (UHM) kinase 1 | −1.53 |

TABLE 3 (c)
DNA Damage/DNA Repair

| | | P-t Fail vs. |
|---|---|---|
| Gene Symbol | Gene Name | CR |
| ADARB1 | adenosine deaminase, RNA-specific, B1 | 2.04 |
| ARHGAP1 | Rho GTPase activating protein 1 | 1.50 |
| ATMIN | ATM interactor | 1.53 |
| BARD1 | BRCA1 associated RING domain 1 | −1.60 |
| CASP9 | caspase 9, apoptosis-related cysteine peptidase | −1.58 |
| CCNB1 | cyclin B1 | −2.05 |
| CDA | cytidine deaminase | 2.52 |
| DTL | denticleless homolog (Drosophila) | −2.75 |
| FANCD2 | Fanconi anemia, complementation group D2 | −1.75 |
| FBXO5 | F-box protein 5 | −1.73 |
| FEN1 | flap structure-specific endonuclease 1 | −1.67 |
| H2AFZ | H2A histone family, member Z | −1.78 |
| HIF1A | hypoxia inducible factor 1, alpha subunit (basic helix-loop- | 1.82 |
| | helix transcription factor) | |
| HJURP | Holliday junction recognition protein | −1.58 |
| HMOX1 | heme oxygenase (decycling) 1 | 2.99 |
| ING4 | inhibitor of growth family, member 4 | −1.51 |
| LAPTM4B | lysosomal protein transmembrane 4 beta | −2.01 |
| LMNB1 | lamin B1 | −1.89 |
| MCM2 | minichromosome maintenance complex component 2 | −2.65 |
| MSH6 | mutS homolog 6 (E. coli) | −1.68 |
| NOX4 | NADPH oxidase 4 | 1.59 |
| PARP1 | poly (ADP-ribose) polymerase 1 | −1.57 |
| PCNA | proliferating cell nuclear antigen | −1.72 |
| PMAIP1 | phorbol-12-myristate-13-acetate-induced protein 1 | −2.27 |
| PTEN | phosphatase and tensin homolog | 1.95 |
| PTTG1 | pituitary tumor-transforming 1 | −2.08 |
| RAD51 | RAD51 homolog (RecA homolog, E. coli) (S. cerevisiae) | −1.68 |
| RAD51AP1 | RAD51 associated protein 1 | −1.78 |
| RAD54B | RAD54 homolog B (S. cerevisiae) | −1.58 |
| RBBP8 | retinoblastoma binding protein 8 | −1.69 |
| RPA2 | replication protein A2, 32 kDa | −1.65 |
| TK1 | thymidine kinase 1, soluble | −1.82 |
| TRIP13 | thyroid hormone receptor interactor 13 | −1.93 |
| TTK | TTK protein kinase | −1.79 |
| YEATS4 | YEATS domain containing 4 | −1.70 |

Table 3 (a) (b) (c) Differentially expressed genes (ANOVA p≦0.05 and 1.5-fold cutoff) between Post-treatment Failures and Complete Responders associated with specific categories of: “Genome Stability/Genetic Instability/Genome Instability,” “Mitosis/Cell Cycle,” and “DNA Damage/DNA Repair.”

Post-treatment samples in patients that failed treatment (Post-Tx Fails) were compared with pre-treatment samples of the complete responders (CRs). The rationale for this study design was that no treatment failures were available in the CR group, and that the group of patients that had recurrences were referred to us from outside institutions, where no pre-treatment biopsies had been obtained.

In certain embodiments, the methods involve measuring a biological sample from a patient. The levels of mRNA or protein expression from at least one or more biomarkers is measured. A biological sample of a head or neck cancer patient is typically assayed. A “biological sample” includes a sample from a tumor, a cancerous tissue, a pre-cancerous tissue, a biopsy, blood, serum, saliva, or a tissue, etc. obtained from a patient suffering from a head or neck cancer or who has yet to be diagnosed with a head or neck cancer. The biological sample is then typically assayed to detect and measure the presence of one or more expression products of the biomarker gene.

Specific mRNA biomarkers may be detected and/or quantified with any detection method or technique known in the art, for example, but not limited to, microarrays, NGS, PCR (polymerase chain reaction); RT-PCR (reverse transcriptase-polymerase chain reaction), or qRT-PCR. Methods may detect the presence or absence of a particular mRNA, sequence changes of a particular mRNA, presence of level (i.e., an amount) of a specific mRNA above a detection threshold or a diagnostic threshold correlated with the presence or absence of a disease, or determine the concentration of specific mRNA in a particular sample. In one embodiment, a sample comprising RNA from a biological sample is used directly to measure the mRNA levels of a biomarker. Any method of measuring or quantitating the amount of mRNA in a biological sample can be used. Preferred methods are reliable, sensitive and specific for a particular mRNA used as a biomarker in aspects of the present invention. Methods such as differential display, RNAase protection assays and Northern or Southern blots may be used to quantify mRNA in a biological sample, or indirectly quantify mRNA in a biological sample through amplification and detection of cDNA oligonucleotides (completely or partially) complementary or (completely or partially) identical to the mRNA biomarker. More recently developed techniques such as qRT-PCR offer more sensitive and less labor-intensive quantification of mRNA in samples. mRNA may or may not be amplified by techniques such as polymerase chain reaction (PCR) prior to measurement, or the quantity of mRNA may be directly or indirectly measured during amplification. Quantitative assessments such as real-time quantitative PCR (qRT-PCR) assay are simple, sensitive, reproducible, and cost-effective; hence they are suitable to use as a diagnostic tool. Example amplification and detection techniques are described below.

For purposes of detection and subsequent quantification, the concentration of mRNA, or cDNA amplification products, may be amplified as part of the detection and amplification process. In many amplification processes, amplification proceeds in a predictable fashion over time, such that final concentrations of amplification products can be used to determine initial concentration of particular mRNA in a sample, prior to amplification.

Several exemplary methods exist for amplifying mRNA nucleic acid sequences such as mature mRNAs, precursor mRNAs, and primary polynucleotides. Suitable nucleic acid polymerization and amplification techniques include reverse transcription (RT), polymerase chain reaction (PCR), real-time PCR (quantitative PCR (q-PCR), nucleic acid sequence-base amplification (NASBA), ligase chain reaction, multiplex ligatable probe amplification, invader technology (Third Wave), rolling circle amplification, in-vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR) (See, e.g., Chen et al., Nucleic Acids Research, 33(20):e179 (2005); Benes and Castoldi, Methods 50(2010), pg 244-249).

A typical PCR reaction includes multiple amplification steps, or cycles that selectively amplify target nucleic acid species: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature mRNAs are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In one particular embodiment, RNA is obtained from a biological sample. The RNA is then transformed into cDNA (complementary DNA) copy using methods known in the art. In particular embodiments, the cDNA is labeled with a fluorescent label or other detectable label. The cDNA is then hybridized to a substrate containing a one or more probes of interest. A probe of interest typically hybridizes under stringent hybridization conditions to the DNA sequence of interest. In certain embodiments, one or more nucleic acid probes are capable of hybridizing to the sequences of interest (e.g., any of SEQ ID NOS: 1-6 or fragments thereof (e.g., fragments are at least about 15 nucleotides in length)) under the hybridization conditions of 6×SSC (0.9 M NaCl, 0.09 M sodium citrate, pH 7.4) at 65° C. The probes may comprise nucleic acids. An example of a nucleic acid is DNA. The term “nucleic acid” refers to deoxyribonucleotides or ribonucleotides and polymers thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and peptide-nucleic acids (PNAs). In certain cases, the probes will be from about 15 to about 50 base pairs in length. The amount of cDNA hybridization can be measured by assaying for the presence of the detectable label, such as a fluorophore. The amount of the hybridization signal can be used to generate a qualitative or quantitative measurement of the level of a nucleic acid of interest in sample. The term “detectable label” refers to a moiety that is attached through covalent or non-covalent means to an entity being measured or a probe. A “detectable label” can be a radioactive moiety, a fluorescent moiety, a chemiluminescent moiety, etc. The term “fluorescent label” refers to label that accepts radiant energy of one wavelength and emits radiant energy of a second wavelength. The presence of a detectable label may be assayed using methods known in the art that are appropriate to detect a particular label, such as spectrophotometric means (e.g., a spectrophotometer), radiometric means (e.g., scintillation counter), fluorometer, luminometer, etc.

Included within the scope of the discovery are DNA microarrays containing a plurality of sequences that hybridize under stringent hybridization conditions to one or more biomarker gene sequences. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 bp of contiguous DNA. In certain embodiments, the DNA probes are from about 20 to about 50 bp of contiguous DNA.

In certain embodiments, the levels of biomarker protein is measured. The levels of biomarker protein may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art. In addition, immunoassays, including immunohistochemistry, may be employed. In certain embodiments, the present invention relates to kits which comprise reagents (such as antibodies) capable of specifically binding to any of the biomarker and its directions for its use. The kit may comprise a container which comprises one or more reagents and directions for their use. Furthermore, mass spectrometry may be used to detect proteins or fragments thereof, and may be used in combination with other techniques such as HPLC.

In another aspect, the measurement of a biomarker(s) of the present invention is used to predict a patient's response to treatment. In one embodiment, in a patient suffering from a virally-induced head or neck cancer (HNSCC), a biomarker measurement can be used to predict the patient's response to treatment. The prediction is based on whether the protein, mRNA, or cDNA levels of the measured biomarker(s) indicate biomarker levels are upregulated, or downregulated, or unchanged. Based on the predication, a particular therapy or therapies may be recommended, not recommended, prioritized, accelerated, or otherwise altered from traditional HNSCC treatment. For example, HNSCC treatment de-escalation could be targeted to patients most likely to benefit from the traditional regimen, and those who would not respond well could be better served by alternate treatment modalities. For those patients that are not expected to respond well to traditional treatment, then alternatives such as minimally invasive surgery, i.e. Transoral lasermicro surgery (TLM) or Transoral robotic surgery (TORS), along with adjuvant radiation with or without chemotherapeutic agents, could be used to give these patients a better chance at treatment success.

In another aspect, the prediction is used to treat a HNSCC patient. The treatment of head and neck cancer in certain embodiments, involves measuring the levels of mRNA or protein of one or more biomarkers selected from the markers identified herein; recommending a particular treatment or treatments for the patient based on the mRNA or protein levels; and providing the recommended treatment. The method of treatment typically further comprises administering a therapeutically effective amount of one or more cancer treatment agents selected from the group consisting of: cancer chemotherapeutic agents and radiation. The treatment of cancer may also comprise surgery or surgical procedures. The term “administering” refers to the method of contacting a compound with a subject. Modes of “administering” may include but are not limited to, methods that involve contacting the cancer chemotherapeutic agents intravenously, intraperitoneally, intranasally, transdermally, topically, via implantation, subcutaneously, parentally, intramuscularly, orally, systemically, and via adsorption. The term “treatment” includes the acute or prophylactic diminishment or alleviation of at least one symptom or characteristic associated or caused by the cancer being treated. For example, treatment can include diminishment of several symptoms of a cancer or complete eradication of a cancer. The phrase “therapeutically effective amount” means an amount of a cancer chemotherapeutic agent, or a pharmaceutically acceptable salt thereof, that is sufficient to inhibit, halt, or allow an improvement in the cancer being treated when administered alone or in conjunction with another pharmaceutical agent or treatment in a particular subject or subject population. For example in a human a therapeutically effective amount can be determined experimentally in a clinical setting, for the particular disease and subject being treated. It should be appreciated that determination of proper dosage forms, dosage amounts and routes of administration is within the level of ordinary skill in the pharmaceutical and medical arts.

Any licensed practitioner would be able to select an appropriate therapeutic regimen. Therapeutic regimens may be comprised of the use of cancer chemotherapeutic agents and/or radiation. Chemoradiotherapy is the use of both radiation and chemotherapy to treat a patient suffering from a cancer. The radiation and chemotherapy do not have to occur simultaneously and can be separated in time, for example by hours, days, or months, etc. A cancer chemotherapeutic agent is a chemical or biological agent (e.g., antibody, protein, RNA, DNA, etc.) that retards, slows, or stops the growth of cancer or is approved to treat a cancer by the U.S. Food and Drug Administration. Examples of head and neck cancer chemotherapeutic agents include, but are not limited to cisplatin, cetuximab, docetaxel, and erlotinib. In particular cases, the chemoradiotherapy comprises administering cisplatin and 5-fluorouracil. Another example of a cancer treatment agent is radiation. In certain embodiments, the cancer is a head or neck cancer. Examples of head or neck cancer include, but are not limited to: squamous cell carcinoma of the head and neck. Further examples of head and neck cancers include oropharyngeal and laryngeal squamous cell carcinoma.

As primary treatment of HNSCC has shifted towards the use of radiotherapy or chemoradiotherapy, predicting tumor response to both modalities is useful in tailoring patient specific therapy. Treatment response to therapy was found to be independent of TNM stage in this study and patients with an incomplete response to their primary therapy (RT or CRT) were more likely to experience distant metastasis and have lower CSS than those with complete response. Therapy can be guided by evaluating individual tumor biology by assessing established prognostic and predictive biomarkers; this approach is already in use in early stage breast cancer to determine the benefit of chemotherapy. Previous work has found low c-met expression to be associated with cisplatin sensitivity, and during the previous study concluded patients with tumors having high expression of c-met may not be good candidates for concomitant chemoradiation (Akervall et al. (2004)). This has been validated as in this study, c-met was significant in predicting for poor response to radiotherapy or chemoradiotherapy. MET is a tyrosine kinase receptor involved in proliferation, mitogenesis, angiogenesis, and metastasis. Overexpression of Met has been reported in breast, ovarian, thyroid, pancreatic, brain, and gastrointestinal tumors, and c-met overexpression has been correlated with poor prognosis in nasopharyngeal carcinoma patients (Qian et al. (2002)). Overexpression of c-met has also been shown to be a predictor of local recurrence in oral tongue carcinoma (Endo et al. (2006)). Currently, c-met was not predictive in CSS. There was no prediction of c-met in RFS; this may due to the fact that patients who had an incomplete response were censored in the RFS analysis.

Having now generally described the invention, the same will be more readily understood through reference to the following examples, which are provided by way of illustration, and are not intended to be limiting of the present invention, unless specified.

### EXAMPLES

### Example 1

### Patients, Materials and Methods

The expression of biomarkers, 1, 2, 3, 4, 5, 6, were correlated from samples taken from pre-treatment patient biopsies with clinical outcomes in HPV-positive HNSCC patients. The patients, specimens, materials and methods are described.

Patients and Specimens

Samples were obtained from pre-treatment biopsies and salvage surgery specimens from patients managed through The Multidisciplinary Head and Neck Clinic at William Beaumont Hospital, Royal Oak, Mich., a tertiary cancer care center. Patients were consented by Beaumont BioBank clinical staff using an IRB approved protocol (HIC 2008-180), and samples were processed and stored at −80° C. using standard operating procedures until required for further analysis. The vast majority of samples were processed and stored within 30 minutes. Table 1 describes the patient and lesion characteristics for the samples used herein.

RNA Isolation.

RNA was isolated from fresh frozen or OTC-preserved tissue using RNeasy™ mini or micro kits (Qiagen). Tissue was minced and then homogenized into RLT buffer using a polytron tissue homogenizer (Thermo Fisher Scientific Inc., Waltham, Mass.) and subsequent passage through a Qiashredder (Qiagen, Valencia, Calif.) column. Following column purification, RNA was quantitated using a Nanodrop spectrophotometer (Thermo Fisher Scientific Inc., Waltham, Mass.) and quality determined by analysis on an Agilent Bioanalyzer (Agilent Technologies Inc., Santa Clara, Calif.). RNA integrity numbers of isolated RNA ranged from 3.1 to 8.2, indicating the range of extracted RNA quality.

HPV16 Screening.

Given that testing for HPV is not a routine clinical procedure, HPV status was determined using a PCR-based (Eppendorf Realplex Mastercycler; Hauppauge, N.Y.) screening method. In order for a patient to be deemed HPV-positive, the E7 gene specific to HPV16 had to be detected in both the DNA and RNA. Due to limited quantities, RNA had to undergo a preliminary amplification step. Total RNA was reverse transcribed using a Superscript III cDNA synthesis kit (Invitrogen; Grand Island, N.Y.) following manufacturer's protocol. cDNA was then pre-amplified using TaqMan PreAmp Master Mix Kit (Applied Biosystems; Grand Island, N.Y.) with a pool of GAPDH and HPV16-E7 primers. The conditions for the pre-amplification were: 95° C. for 10 minutes then 14 cycles of 95° C. for 15 seconds/60° C. for 4 minutes. Pre-amplified samples were then diluted and subject to PCR amplification. The PCR reaction mixture was prepared containing substrate (gDNA or cDNA), TaqMan Gene Expression Master Mix (Applied Biosystems; Grand Island, N.Y.), and either HPV16 E7 or GAPDH primers. The following thermocycling conditions were used: 50° C. for 2 minutes, 95° C. for 2 minutes, then 40 amplification cycles of 95° C. for 15 seconds/60° C. for 1 minute. The following primers and probes were used for HPV16 E7⁸ (Integrated DNA Technologies Inc., San Diego, Calif.): 5′-CGAATGTCTACGTGTGTGCTTTG-3′ (SEQ ID NO. 13), 5′-CCGGACAGAGCCCATTACAA-3′ (SEQ ID NO 14), and probe 5′-CGCACAACCGAAGCGTAGAGTCACACT-3′ (SEQ ID NO 15). Primers and probes for GAPDH were ordered as predesigned assays (Assay Hs99999905_m1 and Assay Hs03929097_g1; Applied Biosystems, Grand Island, N.Y.).

Sample and Microarray Preparation.

Total RNA from each sample was labeled using the Ambion WT Expression Kit (Ambion Inc., Austin, Tex.), which uses a reverse transcription priming method that specifically primes non-ribosomal RNA and provides complete and unbiased coverage of the transcriptome while significantly reducing the priming of rRNA. End labeling was performed according to the Affymetrix GeneChip® WT terminal labeling and hybridization protocol, and Human Exon 1.0 ST Arrays (Affymetrix, Santa Clara, Calif.) were hybridized at 45° C. for 16 hours in a rotating hybridization oven. Arrays were washed and stained on a GeneChip® Fluidics Station 450 using standard protocols for Exon 1.0 ST arrays. Washed and stained arrays were scanned using a GeneChip®, Affymetrix 3000 7G scanner. Resulting CEL files were subject to analysis.

Gene Expression Analysis.

The CEL files containing the raw intensity data from the Affymetrix GeneChip arrays were imported into Partek Genomics Suite (version 6.6beta, build 6.12.0207; Partek Inc., St. Louis, Mo.) and normalized using the robust multichip average with a guanine-cytosine content background correction, quantile normalization, log2-transformation, and median polish probeset summarization. Exons were then summarized to genes using the average of the probesets. Differentially expressed genes were identified using ANOVA (p≦0.05) with two factors: tumor grade and patient (random effect). Hierarchical clustering was carried out using Partek® software, version 6.6 Copyright 2012 (Partek Inc., St. Louis, Mo.). Hierarchical clustering analysis was performed using Euclidean distance as similarity measure and average linkage for the agglomerative method. Gene set enrichment analysis (GSEA) and pathway analysis were performed using Pathway Studio 7.1 (Ariadne Genomics, Rockville, Md.).

Quantitative Real-Time PCR.

Gene expression levels were quantified using a Realplex Mastercycler (Eppendorf, Hauppauge, N.Y.). The following pre-designed TaqMan Gene specific primers (Applied Biosystems, Grand Island, N.Y.) were used: LCE3D (Assay ID Hs00754375_s1), KRTDAP (Assay ID Hs00415563_m1), HMOX1 (Assay ID Hs01110250_m1), KRT19 (Assay ID Hs00761767_s1), MDK (Assay ID Hs00171064_m1), TSPAN1 (Assay ID Hs00371661_m1), and GAPDH (Assay ID Hs99999905_m1). Assay was performed on cDNA generated by the WT amplification of RNA described above. Quantitative real-time PCR reaction mixture was prepared containing 1 ul cDNA (87.6 ng), 1× TaqMan Gene Expression Master Mix (Applied Biosystems, Grand Island, N.Y.), and 1× Gene Expression Assay (Applied Biosystems, Grand Island, N.Y.). The following thermocycling condition was used: 50° C. for 2 min, 95° C. for 10 min, and 40 amplification cycles of 95° C. for 15 seconds/60° C. for 1 min. The delta CT method was used for analysis. For fold-change calculations, CT values were limited to a maximum of 40.

### Example 2

### HPV Testing and Patient Characteristics

DNA and RNA isolated from each sample were analyzed with RT-PCR using primers specific to the E7 gene of HPV16. Nineteen samples were positive for the E7 gene at both the DNA and RNA level. All 19 patient samples were included in subsequent analysis.

Patient demographics and lesion characteristics are included in Table 1. Patients were grouped into Complete Responders (CRs) and Post-treatment Failures (Post-Tx Fails) based upon the patient's response 3 months following chemoradiation therapy. The average age of the CRs (64.3) and the Post-Tx Fails (62.0) are not significantly different. The primary site of all CRs was the oropharynx. The Post-Tx Fails were predominantly situated in the oropharynx, but 2 of the 7 (29%) were in the oral cavity. Both the CRs and Post-Tx Fails ranged from Stage 2-4. Seven of 12 (58%) CRs demonstrated positive lymph nodes, while 3 of the 7 (43%) Post-Tx Fails had positive nodes at time of recurrence.

### Example 3

### Gene Expression Changes in HPV-Positive HNSCC

The 19 samples of HPV-positive head and neck cancer were analyzed using the Human Exon 1.0 ST array from Affymetrix. There are 262 genes identified as showing significant changes in expression (p≦0.05 and a 1.5-fold cutoff) between Post-Tx Fails and CRs, including upregulation of 114 genes in the Post-Tx Fails and downregulation of 148. See Table 4. The most highly upregulated genes included late cornified envelope 3D (LCE3D), keratinocyte differentiation-associated protein (KRTDAP), and heme oxygenase 1 (HMOX1). Among the most downregulated in Post-Tx Fails are tetraspanin 1 (TSPAN1), midkine (MDK, also known as neurite growth-promoting factor 2), and keratin 19 (KRT19).

TABLE 4
Genes Identified as Differentially Expressed between Post-treatment
Failures and Complete Responders

| | | | Fold- | |
|---|---|---|---|---|
| | | | Change | |
| | | | (P-t | |
| | Gene | | Fail | |
| RefSeq | Symbol | Gene Name | vs.CR) | p-value |
| NM_032563 | LCE3D | late cornified envelope 3D | 12.78 | 3.40E−03 |
| NM_207392 | KRTDAP | keratinocyte differentiation- | 5.68 | 1.46E−02 |
| | | associated protein | | |
| NM_006121 | KRT1 | keratin 1 | 4.36 | 2.91E−02 |
| NM_144505 | KLK8 | kallikrein-related peptidase 8 | 3.08 | 2.24E−02 |
| NM_002133 | HMOX1 | heme oxygenase (decycling) 1 | 2.99 | 1.79E−02 |
| NM_032849 | C13orf33 | chromosome 13 open reading frame | 2.86 | 1.38E−02 |
| | | 33 | | |
| NM_005708 | GPC6 | glypican 6 | 2.82 | 2.53E−02 |
| NM_002445 | MSR1 | macrophage scavenger receptor 1 | 2.66 | 2.23E−02 |
| NM_002581 | PAPPA | pregnancy-associated plasma protein | 2.64 | 2.90E−03 |
| | | A, pappalysin 1 | | |
| NM_198148 | CPXM2 | carboxypeptidase X (M14 family), | 2.64 | 1.92E−02 |
| | | member 2 | | |
| NM_004041 | ARRB1 | arrestin, beta 1 | 2.63 | 2.38E−02 |
| NM_004065 | CDR1 | cerebellar degeneration-related | 2.6 | 2.26E−02 |
| | | protein 1, 34 kDa | | |
| NM_001184830 | VSIG4 | V-set and immunoglobulin domain | 2.58 | 4.92E−02 |
| | | containing 4 | | |
| NM_004791 | ITGBL1 | integrin, beta-like 1 (with EGF-like | 2.57 | 1.45E−02 |
| | | repeat domains) | | |
| NM_001785 | CDA | cytidine deaminase | 2.52 | 4.81E−02 |
| NM_001135057 | LRRC15 | leucine rich repeat containing 15 | 2.46 | 1.56E−02 |
| NM_017791 | FLVCR2 | feline leukemia virus subgroup C | 2.45 | 1.83E−02 |
| | | cellular receptor family, member 2 | | |
| NM_006988 | ADAMTS1 | ADAM metallopeptidase with | 2.44 | 3.34E−02 |
| | | thrombospondin type 1 motif, 1 | | |
| AL512709 | TBC1D16 | TBC1 domain family, member 16 | 2.41 | 3.21E−02 |
| NM_005014 | OMD | osteomodulin | 2.34 | 9.60E−03 |
| NR_024042 | GUCY2E | guanylate cyclase 2E | 2.29 | 2.19E−02 |
| NM_031476 | CRISPLD2 | cysteine-rich secretory protein LCCL | 2.23 | 4.82E−02 |
| | | domain containing 2 | | |
| NM_004378 | CRABP1 | cellular retinoic acid binding protein 1 | 2.21 | 3.95E−02 |
| NM_013451 | MYOF | myoferlin | 2.19 | 8.92E−05 |
| NM_203403 | C9orf150 | chromosome 9 open reading frame | 2.16 | 2.10E−02 |
| | | 150 | | |
| NM_144665 | SESN3 | sestrin 3 | 2.15 | 4.21E−02 |
| NM_000259 | MYO5A | myosin VA (heavy chain 12, myoxin) | 2.1 | 1.55E−02 |
| NM_020980 | AQP9 | aquaporin 9 | 2.09 | 1.66E−02 |
| NM_015833 | ADARB1 | adenosine deaminase, RNA-specific, | 2.04 | 2.43E−03 |
| | | B1 | | |
| NM_018404 | ADAP2 | ArfGAP with dual PH domains 2 | 2.04 | 3.02E−02 |
| NM_001080512 | BICC1 | bicaudal C homolog 1 (Drosophila) | 2.03 | 4.62E−02 |
| NM_017565 | FAM20A | family with sequence similarity 20, | 2.02 | 5.04E−03 |
| | | member A | | |
| NM_030817 | APOLD1 | apolipoprotein L domain containing 1 | 2.02 | 3.54E−02 |
| NM_173475 | DCUN1D3 | DCN1, defective in cullin neddylation | 2.02 | 9.61E−03 |
| | | 1, domain containing 3 (S | | |
| NM_080491 | GAB2 | GRB2-associated binding protein 2 | 2 | 7.89E−03 |
| NM_004952 | EFNA3 | ephrin-A3 | 1.98 | 3.54E−02 |
| NM_005729 | PPIF | peptidylprolyl isomerase F | 1.96 | 2.23E−02 |
| NM_000314 | PTEN | phosphatase and tensin homolog | 1.95 | 2.35E−02 |
| NM_001078171 | FAM127A | family with sequence similarity 127, | 1.94 | 1.72E−02 |
| | | member A | | |
| NM_000693 | ALDH1A3 | aldehyde dehydrogenase 1 family, | 1.9 | 4.66E−02 |
| | | member A3 | | |
| NM_001099781 | GGT5 | gamma-glutamyltransferase 5 | 1.9 | 3.31E−02 |
| NM_148897 | SDR9C7 | short chain dehydrogenase/reductase | 1.89 | 4.05E−02 |
| | | family 9C, member 7 | | |
| NM_198277 | SLC37A2 | solute carrier family 37 (glycerol-3- | 1.87 | 4.02E−02 |
| | | phosphate transporter), member 2 | | |
| NM_183239 | GSTO2 | glutathione S-transferase omega 2 | 1.87 | 2.01E−02 |
| NM_001080509 | TSPAN11 | tetraspanin 11 | 1.86 | 2.68E−02 |
| NM_019035 | PCDH18 | protocadherin 18 | 1.85 | 4.76E−02 |
| NM_004969 | IDE | insulin-degrading enzyme | 1.84 | 2.42E−02 |
| NM_003734 | AOC3 | amine oxidase, copper containing 3 | 1.84 | 3.54E−02 |
| NM_001530 | HIF1A | hypoxia inducible factor 1, alpha | 1.82 | 2.20E−02 |
| | | subunit | | |
| NM_021570 | BARX1 | BARX homeobox 1 | 1.82 | 3.09E−02 |
| NM_021033 | RAP2A | RAP2A, member of RAS oncogene | 1.81 | 1.53E−02 |
| | | family | | |
| NM_033397 | ITPRIP | inositol 1,4,5-triphosphate receptor | 1.81 | 2.11E−02 |
| | | interacting protein | | |
| NM_001008707 | EML1 | echinoderm microtubule associated | 1.79 | 4.95E−02 |
| | | protein like 1 | | |
| NM_016046 | EXOSC1 | exosome component 1 | 1.77 | 4.38E−02 |
| NM_178169 | RASSF3 | Ras association (RalGDS/AF-6) | 1.71 | 5.97E−03 |
| | | domain family member 3 | | |
| NM_020650 | RCN3 | reticulocalbin 3, EF-hand calcium | 1.7 | 3.62E−02 |
| | | binding domain | | |
| NM_003952 | RPS6KB2 | ribosomal protein S6 kinase, 70 kDa, | 1.7 | 5.26E−03 |
| | | polypeptide 2 | | |
| NM_182491 | ZFAND2A | zinc finger, AN1-type domain 2A | 1.69 | 2.46E−02 |
| NM_012124 | CHORDC1 | cysteine and histidine-rich domain | 1.69 | 2.77E−02 |
| | | (CHORD)-containing 1 | | |
| NM_017787 | C10orf26 | chromosome 10 open reading frame | 1.67 | 1.21E−02 |
| | | 26 | | |
| NM_002663 | PLD2 | phospholipase D2 | 1.67 | 4.23E−02 |
| NR_023356 | MTMR2 | myotubularin related protein 2 | 1.67 | 9.75E−03 |
| NM_001299 | CNN1 | calponin 1, basic, smooth muscle | 1.66 | 4.97E−02 |
| NM_024099 | C11orf48 | chromosome 11 open reading frame | 1.66 | 4.15E−02 |
| | | 48 | | |
| NM_024628 | SLC12A8 | solute carrier family 12 | 1.65 | 4.57E−02 |
| | | (potassium/chloride transporters), | | |
| | | member 8 | | |
| NM_004996 | ABCC1 | ATP-binding cassette, sub-family C | 1.65 | 1.80E−02 |
| | | (CFTR/MRP), member 1 | | |
| NM_024756 | MMRN2 | multimerin 2 | 1.64 | 4.59E−02 |
| NM_015507 | EGFL6 | EGF-like-domain, multiple 6 | 1.63 | 2.48E−02 |
| NM_138440 | VASN | vasorin | 1.63 | 3.46E−02 |
| NM_001127205 | HMOX2 | heme oxygenase (decycling) 2 | 1.63 | 4.71E−02 |
| NR_033759 | ATP5L | ATP synthase, H+ transporting, | 1.63 | 3.51E−02 |
| | | mitochondrial Fo complex, subunit G | | |
| NM_015221 | DNMBP | dynamin binding protein | 1.63 | 1.85E−02 |
| NM_052875 | VPS26B | vacuolar protein sorting 26 homolog | 1.62 | 7.40E−03 |
| | | B (S. pombe) | | |
| NM_002616 | PER1 | period homolog 1 (Drosophila) | 1.62 | 3.42E−02 |
| NM_198794 | MAP4K5 | mitogen-activated protein kinase | 1.61 | 4.00E−03 |
| | | kinase kinase kinase 5 | | |
| NM_017907 | C11orf59 | chromosome 11 open reading frame | 1.6 | 4.61E−02 |
| | | 59 | | |
| NM_153261 | TMEM188 | transmembrane protein 188 | 1.6 | 1.82E−02 |
| NM_001037343 | CDKL5 | cyclin-dependent kinase-like 5 | 1.6 | 3.01E−02 |
| NM_001025579 | NDEL1 | nudE nuclear distribution gene E | 1.59 | 1.80E−03 |
| | | homolog (A. nidulans)-like 1 | | |
| NM_016931 | NOX4 | NADPH oxidase 4 | 1.59 | 4.43E−02 |
| NM_024652 | LRRK1 | leucine-rich repeat kinase 1 | 1.59 | 2.44E−02 |
| NM_014431 | KIAA1274 | KIAA1274 | 1.59 | 4.54E−02 |
| NM_001172509 | SATB2 | SATB homeobox 2 | 1.59 | 4.38E−02 |
| NM_001144058 | NTM | neurotrimin | 1.59 | 3.19E−02 |
| NM_052950 | WDFY2 | WD repeat and FYVE domain | 1.59 | 1.05E−02 |
| | | containing 2 | | |
| NM_130464 | NPIPL3 | nuclear pore complex interacting | 1.58 | 4.29E−02 |
| | | protein-like 3 | | |
| NM_019087 | ARL15 | ADP-ribosylation factor-like 15 | 1.58 | 4.95E−02 |
| NM_015194 | MYO1D | myosin ID | 1.57 | 3.42E−02 |
| NM_001128159 | VPS53 | vacuolar protein sorting 53 homolog | 1.56 | 3.64E−02 |
| | | (S. cerevisiae) | | |
| NM_032587 | CARD6 | caspase recruitment domain family, | 1.56 | 1.93E−02 |
| | | member 6 | | |
| NM_001168385 | ALG13 | asparagine-linked glycosylation 13 | 1.56 | 4.61E−03 |
| | | homolog (S. cerevisiae) | | |
| NR_027948 | ERC1 | ELKS/RAB6-interacting/CAST | 1.55 | 2.20E−02 |
| | | family member 1 | | |
| NM_004691 | ATP6V0D1 | ATPase, H+ transporting, lysosomal | 1.55 | 4.08E−02 |
| | | 38 kDa, V0 subunit d1 | | |
| NM_024116 | TAF1D | TATA box binding protein (TBP)- | 1.55 | 1.58E−02 |
| | | associated factor, RNA polymerase I, | | |
| | | D, 41 kDa | | |
| NM_020698 | TMCC3 | transmembrane and coiled-coil | 1.54 | 2.06E−02 |
| | | domain family 3 | | |
| NM_006364 | SEC23A | Sec23 homolog A (S. cerevisiae) | 1.54 | 4.39E−02 |
| NM_001136154 | ERG | v-ets erythroblastosis virus E26 | 1.54 | 3.32E−03 |
| | | oncogene homolog (avian) | | |
| NM_001002915 | IGFL2 | IGF-like family member 2 | 1.54 | 3.82E−02 |
| NM_015346 | ZFYVE26 | zinc finger, FYVE domain | 1.53 | 1.31E−02 |
| | | containing 26 | | |
| NM_203351 | MAP3K3 | mitogen-activated protein kinase | 1.53 | 1.35E−02 |
| | | kinase kinase 3 | | |
| NM_004832 | GSTO1 | glutathione S-transferase omega 1 | 1.53 | 4.76E−02 |
| NM_006195 | PBX3 | pre-B-cell leukemia homeobox 3 | 1.53 | 3.11E−02 |
| NM_033306 | CASP4 | caspase 4, apoptosis-related cysteine | 1.53 | 1.81E−02 |
| | | peptidase | | |
| NM_015251 | ATMIN | ATM interactor | 1.53 | 1.89E−02 |
| NM_001142864 | FAM38A | family with sequence similarity 38, | 1.52 | 3.77E−02 |
| | | member A | | |
| NM_032322 | RNF135 | ring finger protein 135 | 1.52 | 5.70E−03 |
| NM_001896 | CSNK2A2 | casein kinase 2, alpha prime | 1.51 | 3.15E−02 |
| | | polypeptide | | |
| NM_016106 | SCFD1 | sec1 family domain containing 1 | 1.51 | 2.17E−02 |
| NM_032776 | JMJD1C | jumonji domain containing 1C | 1.51 | 2.25E−04 |
| NM_013309 | SLC30A4 | solute carrier family 30 (zinc | 1.51 | 3.84E−02 |
| | | transporter), member 4 | | |
| NM_153021 | PLB1 | phospholipase B1 | 1.51 | 2.18E−03 |
| NM_152545 | RASGEF1B | RasGEF domain family, member 1B | 1.51 | 4.90E−02 |
| NR_002775 | RPLP0P2 | ribosomal protein, large, P0 | 1.51 | 1.69E−02 |
| | | pseudogene 2 | | |
| NM_004308 | ARHGAP1 | Rho GTPase activating protein 1 | 1.5 | 4.86E−02 |
| NM_181644 | MFSD4 | major facilitator superfamily domain | −1.5 | 3.35E−02 |
| | | containing 4 | | |
| NM_003472 | DEK | DEK oncogene | −1.5 | 4.35E−02 |
| NM_016162 | ING4 | inhibitor of growth family, member 4 | −1.51 | 3.40E−02 |
| NM_013328 | PYCR2 | pyrroline-5-carboxylate reductase | −1.51 | 1.74E−03 |
| | | family, member 2 | | |
| NM_001168364 | KRTCAP3 | keratinocyte associated protein 3 | −1.51 | 1.96E−02 |
| NM_020935 | USP37 | ubiquitin specific peptidase 37 | −1.52 | 9.38E−03 |
| NM_003526 | HIST1H2BC | histone cluster 1, H2bc | −1.52 | 2.20E−02 |
| NM_175866 | UHMK1 | U2AF homology motif (UHM) | −1.52 | 4.75E−02 |
| | | kinase 1 | | |
| NM_152522 | ARL6IP6 | ADP-ribosylation-like factor 6 | −1.53 | 2.15E−02 |
| | | interacting protein 6 | | |
| NM_001034194 | EXOSC9 | exosome component 9 | −1.53 | 2.77E−02 |
| NM_001130821 | RABL5 | RAB, member RAS oncogene | −1.53 | 4.09E−02 |
| | | family-like 5 | | |
| NM_173083 | LIN9 | lin-9 homolog (C. elegans) | −1.54 | 4.38E−02 |
| NM_033026 | PCLO | piccolo (presynaptic cytomatrix | −1.54 | 6.22E−03 |
| | | protein) | | |
| NM_001166417 | SSX2IP | synovial sarcoma, X breakpoint 2 | −1.54 | 1.68E−03 |
| | | interacting protein | | |
| NM_004891 | MRPL33 | mitochondrial ribosomal protein L33 | −1.54 | 3.27E−02 |
| NM_018944 | C21orf45 | chromosome 21 open reading frame | −1.55 | 3.38E−02 |
| | | 45 | | |
| NM_003495 | HIST1H4I | histone cluster 1, H4i | −1.55 | 1.38E−02 |
| NM_012073 | CCT5 | chaperonin containing TCP1, subunit | −1.56 | 4.08E−02 |
| | | 5 (epsilon) | | |
| NM_024102 | WDR77 | WD repeat domain 77 | −1.56 | 8.47E−03 |
| NM_031283 | TCF7L1 | transcription factor 7-like 1 (T-cell | −1.56 | 2.34E−02 |
| | | specific, HMG-box) | | |
| NM_152515 | CKAP2L | cytoskeleton associated protein 2-like | −1.57 | 2.91E−02 |
| NM_001618 | PARP1 | poly (ADP-ribose) polymerase 1 | −1.57 | 4.36E−02 |
| NM_012415 | RAD54B | RAD54 homolog B (S. cerevisiae) | −1.58 | 1.60E−02 |
| NM_000898 | MAOB | monoamine oxidase B | −1.58 | 3.09E−02 |
| NM_001229 | CASP9 | caspase 9, apoptosis-related cysteine | −1.58 | 3.05E−02 |
| | | peptidase | | |
| NM_018410 | HJURP | Holliday junction recognition protein | −1.58 | 5.16E−03 |
| NM_012343 | NNT | nicotinamide nucleotide | −1.6 | 4.99E−02 |
| | | transhydrogenase | | |
| NM_000465 | BARD1 | BRCA1 associated RING domain 1 | −1.6 | 2.46E−02 |
| NM_015143 | METAP1 | methionyl aminopeptidase 1 | −1.6 | 2.81E−02 |
| ENST00000392647 | KLHL23 | kelch-like 23 (Drosophila) | −1.61 | 2.45E−02 |
| NM_001037582 | SCD5 | stearoyl-CoA desaturase 5 | −1.61 | 9.70E−03 |
| NM_006397 | RNASEH2A | ribonuclease H2, subunit A | −1.61 | 1.04E−02 |
| NM_153485 | NUP155 | nucleoporin 155 kDa | −1.62 | 3.93E−02 |
| NM_015028 | TNIK | TRAF2 and NCK interacting kinase | −1.62 | 3.66E−02 |
| NM_001185012 | NDUFA2 | NADH dehydrogenase (ubiquinone) | −1.62 | 1.25E−02 |
| | | 1 alpha subcomplex, 2, 8 kDa | | |
| NM_003389 | CORO2A | coronin, actin binding protein, 2A | −1.64 | 2.27E−02 |
| NM_017576 | KIF27 | kinesin family member 27 | −1.65 | 6.67E−03 |
| NM_005524 | HES1 | hairy and enhancer of split 1, | −1.65 | 3.29E−02 |
| | | (Drosophila) | | |
| NM_002946 | RPA2 | replication protein A2, 32 kDa | −1.65 | 1.40E−02 |
| NM_006754 | SYPL1 | synaptophysin-like 1 | −1.66 | 2.42E−02 |
| NM_004168 | SDHA | succinate dehydrogenase complex, | −1.67 | 3.95E−03 |
| | | subunit A, flavoprotein (Fp) | | |
| NM_001145316 | DSN1 | DSN1, MIND kinetochore complex | −1.67 | 2.59E−02 |
| | | component, homolog (S. cerevisiae) | | |
| NM_004111 | FEN1 | flap structure-specific endonuclease 1 | −1.67 | 2.00E−02 |
| NM_007280 | OIP5 | Opa interacting protein 5 | −1.68 | 3.01E−02 |
| NM_001004309 | ZNF774 | zinc forger protein 774 | −1.68 | 4.59E−02 |
| NM_001130675 | CLGN | calmegin | −1.68 | 2.40E−02 |
| NM_002875 | RAD51 | RAD51 homolog (RecA homolog, E. coli) | −1.68 | 4.71E−03 |
| | | (S. cerevisiae) | | |
| NM_031422 | CHST9 | carbohydrate (N-acetylgalactosamine | −1.68 | 4.27E−02 |
| | | 4-0) sulfotransferase 9 | | |
| NM_138409 | MRAP2 | melanocortin 2 receptor accessory | −1.68 | 3.38E−02 |
| | | protein 2 | | |
| NM_000179 | MSH6 | mutS homolog 6 (E. coli) | −1.68 | 9.38E−03 |
| NM_002894 | RBBP8 | retinoblastoma binding protein 8 | −1.69 | 4.80E−02 |
| NM_145697 | NUF2 | NUF2, NDC80 kinetochore complex | −1.69 | 4.80E−02 |
| | | component, homolog (S. cerevisiae) | | |
| NM_006530 | YEATS4 | YEATS domain containing 4 | −1.7 | 1.94E−03 |
| NM_024329 | EFHD2 | EF-hand domain family, member D2 | −1.7 | 4.16E−02 |
| NM_199187 | KRT18 | keratin 18 | −1.71 | 2.53E−02 |
| NM_001130173 | MYB | v-myb myeloblastosis viral oncogene | −1.71 | 6.05E−03 |
| | | homolog (avian) | | |
| NM_178865 | SERINC2 | serine incorporator 2 | −1.72 | 3.62E−02 |
| NM_001104629 | C4orf19 | chromosome 4 open reading frame | −1.72 | 1.42E−02 |
| | | 19 | | |
| NM_012121 | CDC42EP4 | CDC42 effector protein (Rho | −1.72 | 3.58E−02 |
| | | GTPase binding) 4 | | |
| NM_014279 | OLFM1 | olfactomedin 1 | −1.72 | 6.34E−03 |
| NM_002592 | PCNA | proliferating cell nuclear antigen | −1.72 | 1.52E−02 |
| NM_152487 | TMEM56 | transmembrane protein 56 | −1.72 | 1.13E−02 |
| NM_006347 | PPIH | peptidylprolyl isomerase H | −1.72 | 3.13E−02 |
| | | (cyclophilin H) | | |
| NM_002633 | PGM1 | phosphoglucomutase 1 | −1.72 | 4.38E−02 |
| NM_012177 | FBXO5 | F-box protein 5 | −1.73 | 9.49E−03 |
| NM_004242 | HMGN3 | high mobility group nucleosomal | −1.74 | 3.06E−02 |
| | | binding domain 3 | | |
| NM_016010 | FAM164A | family with sequence similarity 164, | −1.74 | 1.59E−02 |
| | | member A | | |
| NM_002388 | MCM3 | minichromosome maintenance | −1.74 | 3.92E−02 |
| | | complex component 3 | | |
| NM_033084 | FANCD2 | Fanconi anemia, complementation | −1.75 | 2.52E−02 |
| | | group D2 | | |
| NM_000946 | PRIM1 | primase, DNA, polypeptide 1 | −1.75 | 3.80E−02 |
| | | (49 kDa) | | |
| NM_001532 | SLC29A2 | solute carrier family 29 (nucleoside | −1.76 | 1.23E−02 |
| | | transporters), member 2 | | |
| NM_001009566 | CLSTN1 | calsyntenin 1 | −1.76 | 4.10E−02 |
| NM_012446 | SSBP2 | single-stranded DNA binding protein 2 | −1.77 | 4.48E−03 |
| NM_005447 | RASSF9 | Ras association (RalGDS/AF-6) | −1.77 | 1.52E−02 |
| | | domain family (N-terminal) member | | |
| NM_002106 | H2AFZ | H2A histone family, member Z | −1.78 | 1.50E−02 |
| NM_003533 | HIST1H3I | histone cluster 1, H3i | −1.78 | 2.92E−02 |
| NM_001130862 | RAD51AP1 | RAD51 associated protein 1 | −1.78 | 2.40E−02 |
| NM_013262 | MYLIP | myosin regulatory light chain | −1.79 | 4.92E−02 |
| | | interacting protein | | |
| NM_015341 | NCAPH | non-SMC condensin I complex, | −1.79 | 1.12E−02 |
| | | subunit H | | |
| NM_007129 | ZIC2 | Zic family member 2 (odd-paired | −1.79 | 1.87E−02 |
| | | homolog, Drosophila) | | |
| NM_003318 | TTK | TTK protein kinase | −1.79 | 1.68E−02 |
| NM_001172312 | PLS1 | plastin 1 | −1.79 | 3.29E−02 |
| NM_032412 | C5orf32 | chromosome 5 open reading frame | −1.8 | 3.96E−02 |
| | | 32 | | |
| NM_018031 | WDR6 | WD repeat domain 6 | −1.8 | 2.48E−03 |
| NM_003510 | HIST1H2AK | histone cluster 1, H2ak | −1.8 | 4.22E−02 |
| NM_001042550 | SMC2 | structural maintenance of | −1.8 | 4.06E−02 |
| | | chromosomes 2 | | |
| NM_203401 | STMN1 | stathmin 1 | −1.81 | 3.27E−02 |
| NM_003258 | TK1 | thymidine kinase 1, soluble | −1.82 | 4.40E−02 |
| NR_026550 | BRP44 | brain protein 44 | −1.82 | 2.70E−03 |
| NM_015541 | LRIG1 | leucine-rich repeats and | −1.83 | 2.45E−02 |
| | | immunoglobulin-like domains 1 | | |
| NM_178824 | WDR49 | WD repeat domain 49 | −1.83 | 2.58E−03 |
| NM_020796 | SEMA6A | sema domain, transmembrane | −1.85 | 1.27E−02 |
| | | domain (TM), and cytoplasmic | | |
| | | domain, | | |
| NM_002497 | NEK2 | NIMA (never in mitosis gene a)- | −1.87 | 6.28E−03 |
| | | related kinase 2 | | |
| NM_014109 | ATAD2 | ATPase family, AAA domain | −1.89 | 1.89E−02 |
| | | containing 2 | | |
| NM_005573 | LMNB1 | lamin B1 | −1.89 | 2.00E−02 |
| NM_003542 | HIST1H4C | histone cluster 1, H4c | −1.9 | 2.06E−02 |
| NM_001195434 | MLF1 | myeloid leukemia factor 1 | −1.92 | 4.39E−02 |
| NM_004237 | TRIP13 | thyroid hormone receptor interactor | −1.93 | 1.88E−02 |
| | | 13 | | |
| NM_001178138 | TFDP2 | transcription factor Dp-2 (E2F | −1.94 | 4.15E−02 |
| | | dimerization partner 2) | | |
| NM_175065 | HIST2H2AB | histone cluster 2, H2ab | −1.95 | 1.41E−02 |
| NM_080738 | EDARADD | EDAR-associated death domain | −1.96 | 1.76E−03 |
| NM_021067 | GINS1 | GINS complex subunit 1 (Psf1 | −1.96 | 2.01E−02 |
| | | homolog) | | |
| NM_001012410 | SGOL1 | shugoshin-like 1 (S. pombe) | −1.96 | 3.58E−03 |
| NM_021572 | ENPP5 | ectonucleotide | −1.97 | 1.87E−02 |
| | | pyrophosphatase/phosphodiesterase 5 | | |
| | | (putative) | | |
| NM_144576 | COQ10A | coenzyme Q10 homolog A (S. cerevisiae) | −1.97 | 3.12E−02 |
| NM_006823 | PKIA | protein kinase (cAMP-dependent, | −1.98 | 4.31E−02 |
| | | catalytic) inhibitor alpha | | |
| NM_018407 | LAPTM4B | lysosomal protein transmembrane 4 | −2.01 | 4.27E−02 |
| | | beta | | |
| NM_001145303 | TMC4 | transmembrane channel-like 4 | −2.01 | 3.59E−03 |
| NM_001677 | ATP1B1 | ATPase, Na+/K+ transporting, beta 1 | −2.02 | 4.87E−02 |
| | | polypeptide | | |
| NM_005915 | MCM6 | minichromosome maintenance | −2.02 | 1.04E−02 |
| | | complex component 6 | | |
| NM_006192 | PAX1 | paired box 1 | −2.03 | 3.44E−02 |
| NM_031966 | CCNB1 | cyclin B1 | −2.05 | 1.82E−02 |
| NM_005914 | MCM4 | minichromosome maintenance | −2.06 | 2.46E−02 |
| | | complex component 4 | | |
| NM_001105077 | MECOM | MDS1 and EVI1 complex locus | −2.08 | 9.15E−03 |
| NM_004219 | PTTG1 | pituitary tumor-transforming 1 | −2.08 | 2.69E−02 |
| NM_022145 | CENPK | centromere protein K | −2.09 | 4.94E−02 |
| NM_016343 | CENPF | centromere protein F, 350/400 kDa | −2.1 | 4.39E−02 |
| | | (mitosin) | | |
| NM_015068 | PEG10 | paternally expressed 10 | −2.13 | 3.65E−02 |
| NM_014176 | UBE2T | ubiquitin-conjugating enzyme E2T | −2.15 | 1.02E−02 |
| | | (putative) | | |
| NM_002999 | SDC4 | syndecan 4 | −2.16 | 1.18E−02 |
| NM_016353 | ZDHHC2 | zinc finger, DHHC-type containing 2 | −2.21 | 6.41E−03 |
| NM_024923 | NUP210 | nucleoporin 210 kDa | −2.22 | 1.56E−02 |
| NM_006408 | AGR2 | anterior gradient homolog 2 | −2.23 | 4.86E−02 |
| | | (Xenopus laevis) | | |
| NM_003619 | PRSS12 | protease, serine, 12 (neurotrypsin, | −2.26 | 2.80E−03 |
| | | motopsin) | | |
| NM_002203 | ITGA2 | integrin, alpha 2 (CD49B, alpha 2 | −2.26 | 2.22E−03 |
| | | subunit of VLA-2 receptor) | | |
| NM_021127 | PMAIP1 | phorbol-12-myristate-13-acetate- | −2.27 | 4.66E−02 |
| | | induced protein 1 | | |
| NM_005244 | EYA2 | eyes absent homolog 2 (Drosophila) | −2.28 | 2.85E−02 |
| NM_003196 | TCEA3 | transcription elongation factor A | −2.33 | 2.71E−02 |
| | | (SII), 3 | | |
| NM_003944 | SELENBP1 | selenium binding protein 1 | −2.38 | 3.46E−02 |
| NM_021101 | CLDN1 | claudin 1 | −2.4 | 3.67E−02 |
| NM_006765 | TUSC3 | tumor suppressor candidate 3 | −2.4 | 2.27E−02 |
| NM_020647 | JPH1 | junctophilin 1 | −2.44 | 4.37E−02 |
| NM_021018 | HIST1H3F | histone cluster 1, H3f | −2.45 | 4.29E−02 |
| NM_019605 | SERTAD4 | SERTA domain containing 4 | −2.53 | 3.27E−02 |
| NM_003529 | HIST1H3A | histone cluster 1, H3a | −2.55 | 2.73E−02 |
| NM_004526 | MCM2 | minichromosome maintenance | −2.65 | 3.48E−02 |
| | | complex component 2 | | |
| NM_016448 | DTL | denticleless homolog (Drosophila) | −2.75 | 2.69E−02 |
| NM_003544 | HIST1H4B | histone cluster 1, H4b | −2.97 | 1.37E−02 |
| NM_020873 | LRRN1 | leucine rich repeat neuronal 1 | −3.1 | 3.63E−02 |
| NM_002276 | KRT19 | keratin 19 | −3.13 | 2.26E−02 |
| NM_001943 | DSG2 | desmoglein 2 | −3.18 | 3.73E−02 |
| NM_001012334 | MDK | midkine (neurite growth-promoting | −3.47 | 6.21E−03 |
| | | factor 2) | | |
| NM_016140 | TPPP3 | tubulin polymerization-promoting | −3.49 | 2.00E−02 |
| | | protein family member 3 | | |
| NM_006252 | PRKAA2 | protein kinase, AMP-activated, alpha | −3.63 | 3.65E−02 |
| | | 2 catalytic subunit | | |
| NM_006103 | WFDC2 | WAP four-disulfide core domain 2 | −3.68 | 5.57E−03 |
| NM_000782 | CYP24A1 | cytochrome P450, family 24, | −4.19 | 2.21E−02 |
| | | subfamily A, polypeptide 1 | | |
| NM_152997 | C4orf7 | chromosome 4 open reading frame 7 | −5.35 | 3.73E−02 |
| NM_005727 | TSPAN1 | tetraspanin 1 | −5.54 | 7.91E−03 |

Table 4: Genes identified as differentially expressed between Post-treatment Failures and Complete Responders. ANOVA ≦0.05 and 1.5-fold cutoff.

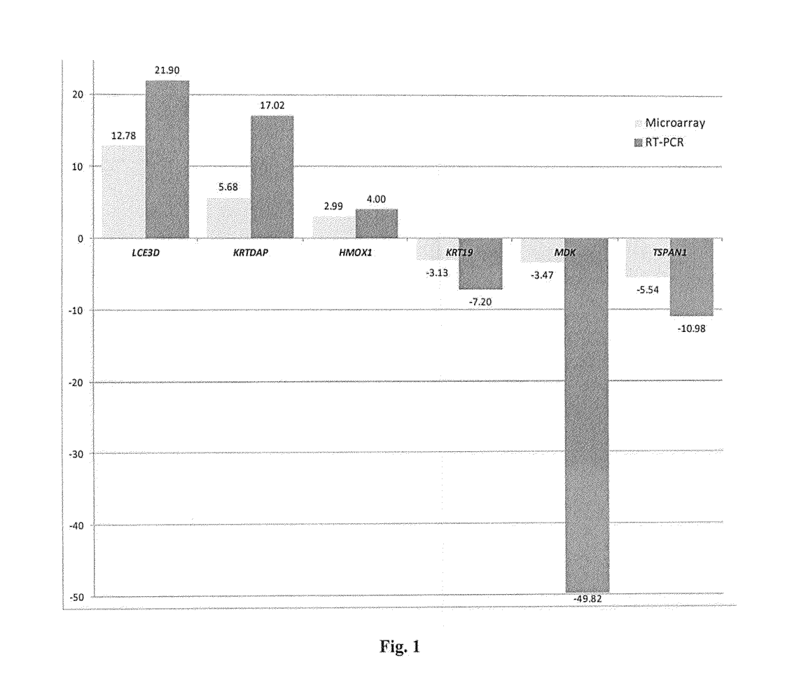

For microarray validation, samples from fourteen patients (7 CRs and 7 Post-Tx Fails) were used to verify the microarray results of these 6 genes. RT-PCR confirmed the microarray results of all 6 genes (FIG. 1). As seen previously, the microarray results tend to underestimate the degree of change found by RT-PCR. MDK showed the greatest disparity between the microarray and RT-PCR results. Given that the fold change is the ratio of the CRs and Post-Tx Fails, the lack of MDK expression in 3 of the 7 Post-Tx Fails by RT-PCR analysis resulted in a distorted representation of fold-change.

Ariadne Pathway Studio® (Ariadne Genomics Inc., Rockville, Md.) was then used to categorize the genes differentially expressed between CRs and Post-Tx Fails in HPV-positive HNSCC. Ariadne constructs sub-networks based upon the results of MedScan® (Ariadne Genomics Inc., Rockville, Md.), a literature mining program that searches publicly available literature such as PubMed for relationships between entities. One such sub-network categorizes genes based upon protein involvement in regulating specific cellular processes. A Fisher's exact test was utilized to determine which cellular process networks were over-represented by entities in the list of 262 differentially expressed genes. Table 2 lists the top 15 cellular processes that are highly regulated between the CRs and Post-Tx Fails. These highly regulated categories consist of two main groups: DNA damage-related (genome stability, genetic instability, genome instability, response to DNA damage, and DNA repair) and cell cycle-related (mitosis, cell cycle checkpoint, cell cycle, S phase, cell proliferation).

### Example 4

### Hierarchical Clustering

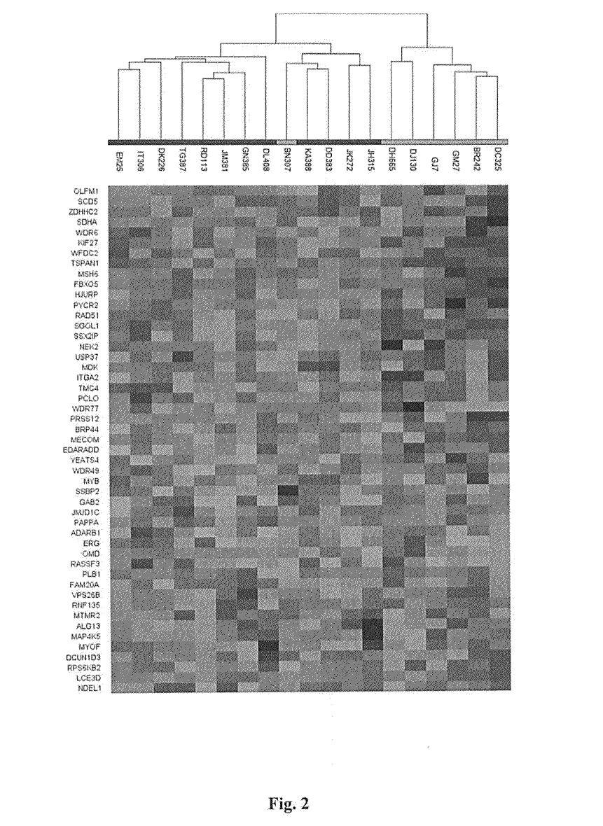

Hierarchical clustering was performed using a subset of these differentially expressed genes. The patients were clustered based upon the 49 genes differentially expressed at p≦0.01 and a 1.5-fold cutoff. These 49 differentially expressed genes adequately delineated between the CRs and the Post-Tx Fails samples (FIG. 2). Only a single Post-Tx Fail patient (BN307) incorrectly clustered with the CRs. The clustering of this patient is difficult to explain given that the patient shows high tumor stage (stage 4A), involvement of contralateral lymph nodes, evidence of alcohol and extensive (100+ pack years) tobacco use, and synchronous lung cancer.

### Example 5

### Sub-Network and Pathway Analysis

In order to better understand the biological context of the changes that differentiate the response of HPV-positive HNSCC to chemoradiation, the microarray data was analyzed using Sub-Network Enrichment Analysis (SNEA) (Ariadne Genomics Inc., Rockville, Md.) to identify expression pathway sub-networks (Nikitin A. et al. Bioinformatics 2003; 19(16):2155-7) (Kotelnikova E. et al. PLoS One 2010; 5(2):e9256.) Expression pathway sub-network analysis consists of a single seed (i.e. gene or functional class) and proteins associated to this seed either by regulating expression of/by the seed or by binding to the promoter of/by the seed (Yuryev A. et al. BMC Bioinformatics 2006; 7:171). Similar to the commonly used Gene Set Enrichment Analysis (Subramanian A. et al. Proc Natl Acad Sci USA 2005; 102(43):15545-50). SNEA interrogates the microarray dataset with no prior significance filtering. Both the level of regulation in the network as well as the size of the network determine enrichment of the sub-network. Three of the most highly regulated sub-networks between CRs and Post-Tx Fails were built around the interrelated seeds of E2F3, E2F4, and the general E2F family. The three E2F-associated sub-networks were combined, and the visualized sub-network was limited to include only those genes that were differentially expressed at ANOVA p≦0.10 and a 1.2-fold cutoff (FIG. 3). Less stringent conditions have previously been shown to be more appropriate for pathway analysis (Benes F M et al. Mol Psychiatry 2006; 11(3):241-51) (Martin M V et al. BMC Med Genomics 2009; 2:62). The combination of these sub-networks illustrates that many of the genes associated with E2 Fare generally downregulated in patients that fail to respond to chemoradiation therapy.

We claim:

1. A method for predicting the response to treatment in a patient and or a method of treating a patient suffering from a head or neck tumor comprising:
a) obtaining a biological sample of said tumor from said patient;
b) measuring one or more of the protein, mRNA, or cDNA levels of at least one biomarker in said sample to obtain a biomarker measurement, wherein said biomarker is selected from the group consisting of
(a) LCE3D;
(b) KRTDAP;
(c) HMOX1;
(d) KRT19;
(e) MDK; and
(f) TSPAN1;
in any combination thereof;
and using the measurement to predict a patient's response to treatment.

2. The method of claim 1 wherein said head or neck tumor is a HNSCC and the HNSCC is a HPV-positive HNSCC and of biomarkers a), b) and/or c), in any combination, show gene upregulation in said sample and, optionally, when any of biomarkers d), e) and/or f), in any combination, show gene downregulation in said sample.

3. The method of claim 1, wherein the protein, mRNA, or cDNA levels are determined by measuring the levels of one or more nucleic acid sequences, or fragments thereof, selected from the group consisting of:
(a)
SEQ ID NO: 1
(LCE3D-cDNA);
(b)
SEQ ID NO: 2
(KRTDAP-cDNA);
(c)
SEQ ID NO: 3
(HMOX1-cDNA);
(d)
SEQ ID NO: 4
(KRT19-cDNA);
(e)
SEQ ID NO: 5
(MDK-cDNA);
(f)
SEQ ID NO: 6
(TSPAN1-cDNA);
(h)
SEQ ID. NO 7
(LCE3D-Nptide);
(i)
SEQ ID NO: 8
(KRTDAP-Peptide);
(j)
SEQ ID NO: 9
(HMOX1-Peptide);
(k)
SEQ ID NO: 10
(KRT19-Peptide);
(m)
SEQ ID NO: 11
(MDK-Peptide);
and
(n)
SEQ ID NO: 12
(TSPAN1-Peptide).
and wherein said fragments are 15, about 15 or greater than about 15 consecutive amino acids or nucleotides.

4. The method of claim 1, wherein said biomarker is selected from a gene from the group consisting of:
(a) LCE3D;
(b) KRTDAP; and
(c) HMOX1;
and using said measurement to predict a response for said patient, the prediction is based on whether the protein, mRNA, or cDNA levels of the measured biomarker(s) indicate biomarker levels are upregulated, or downregulated, or unchanged.

5. The method of claim 4 wherein when said protein, mRNA, or cDNA levels of the measured biomarker(s) are upregulated, and said tumor is identified as HPV-positive HNSCC, then the prediction is that said patient will NOT respond well to traditional therapy and when said protein, mRNA, or cDNA levels of the measured biomarker(s) are NOT upregulated and said tumor is identified as HPV-positive HNSCC, then the prediction is that said patient WILL respond well to traditional therapy.

6. The method of claim 1, wherein said biomarker is selected from a gene selected from the group consisting of:
(d) KRT19;
(e) MDK; and
(f) TSPAN1
and using said measurement to predict a response for said patient, where the prediction is based on whether the protein, mRNA, or cDNA levels of the measured biomarker(s) are upregulated, downregulated, or unchanged.

7. The method of claim 6 wherein when said protein, mRNA, or cDNA levels of the measured biomarker(s) are observed to be DOWNregulated and the prediction is made that the head or neck tumor is one that is HPV-positive HNSCC and said patient will NOT respond well to traditional therapy and when said protein, mRNA, or cDNA levels of the measured biomarker(s) are NOT observed to be DOWNregulated and said tumor is HPV-positive HNSCC, then the prediction is that the patient WILL respond well to traditional therapy.

8. The method of making a treatment recommendation for a patient suffering from a head or neck tumor comprising:
a) obtaining a biological sample of said tumor from said patient;
b) measuring one or more of the protein, mRNA, or cDNA levels of at least one biomarker in said sample to obtain a biomarker measurement, wherein said biomarker is selected from the group consisting of
(a) LCE3D;
(b) KRTDAP;
(c) HMOX1;
(d) KRT19;
(e) MDK; and
(f) TSPAN1;
in any combination thereof;
and using said measurement to make a recommendation for treatment in said patient when any one or more of biomarkers (a-c), or any combination of biomarkers (a-c), indicate gene upregulation in said sample and/or any one or more of biomarkers (d-f), or any combination of biomarkers (d-f), indicate gene downregulation in said sample.

9. The method of claim 8 where the treatment is to provide the patient with non-traditional or alternate treatment modalities that do not include chemoradiation therapy.

10. The method of claims 8 wherein one of the following (a-j) groups of biomarkers are measured:
a) only (1) biomarker is measured;
b) at least two (2) biomarkers are measured;
c) only two (2) biomarkers are measured;
d) at least three (3) biomarkers are measured;
e) only three (3) biomarkers are measured;
f) at least four (4) biomarkers are measured;
g) only four (4) biomarkers are measured;
h) at least five (5) biomarkers are measured;
i) only five (5) biomarkers are measured; or
j) (6) biomarkers are measured.

11. The method of claim 10 wherein the two biomarkers measured are LCE3D and KRTDAP.

12. The method of claim 10 wherein LCE3D is measured.

13. The method of claim 10 wherein KRTDAP is measured.

14. The method for predicting the response to treatment and or a method of making a treatment recommendation, using the procedures of claim 8 wherein said biological sample is from a tumor, a cancerous tissue, a pre-cancerous tissue, a biopsy, blood, serum, saliva, or a tissue.

15. The method of claim 14, wherein said head or neck cancer is head and neck squamous cell carcinoma or HNSCC.

16. The method of claim 15, wherein said cancer is oropharyngeal or laryngeal squamous cell carcinoma.

17. The method of claim 16, wherein said oropharyngeal or laryngeal squamous cell carcinoma is from a squamous cell carcinoma of the head and neck or HNSCC and a prediction is made to determine whether or not said patient will respond well to traditional therapy.

18. A method for prognosis, or evaluation of the effectiveness of a treatment, of a solid tumor in a patient of interest, wherein said tumor is a HNSCC and said method of evaluation comprises obtaining a sample of said HNSCC, then determining whether said patient has a HPV-positive HNSCC, then further determining whether said patient will respond favorably to chemo or radiotherapy using the method for predicting the response to treatment, method of making a treatment recommendation, and the method of treatment is known to one skilled in the art, and includes the option, when the HNSCC is determined to be HPV-positive HNSCC, then the patient is selected for “therapy de-escalation” including treatments that do not include chemoradiation.

19. A method of determining a prognosis of cancer characterized by analysis of a sample comprising the following steps:
a) determine if the sample is HPV-positive HNSCC or HPV-negative HNSCC; and if the sample is HPV-positive HNSCC then;
b) determine whether the patient is likely or not likely to benefit from traditional therapy and wherein said determination is made based on whether or not at least one biomarker from the following group is obtained from the patient's tumor and where the biomarkers is said to be upregulated with biomarkers
(a) LCE3D;
(b) KRTDAP;
(c) HMOX1;
and downregulated in biomarkers selected from the group consisting of (d-f):
(d) KRT19;
(e) MDK; and
(f) TSPAN1
where said biomarkers may be in any number or combination or may be only those as described exactly above as (a)-(f).

20. A method of claim 19 wherein 1 to 6 biomarkers is provided in a kit for prognostic use, in any combination of any number from 1 to 6 comprising:
at least one biomarker selected from the group consisting of a-f; wherein
(a) LCE3D;
(b) KRTDAP;
(c) HMOX1;
(d) KRT19;
(e) MDK; and
(f) TSPAN1.


SEQUENCE LISTING (15 sequences)

SEQ ID NO: 1 - DNA, 617 bp, Homo sapiens
ctcaggaacagccttctcctgcctcctctgcacctggacaactcaactcctgccaagatgtcctgccagcagaaccagcagcagtgccaacccccacccaagtgtccctcacccaagtgtcccccaaagagcccagtacagtgtctgcctccagcttcctctggctgtgccccaagctctgggggctgtggccctagctccgagggcggctgcttcctgaaccaccacaggcgccaccaccgatgccggcgccagaggcccaactcctgtgacaggggcagtggtcagcaaggcgggggctctggctgcggccatggctctggaggctgctgctgacctggatcctgatgctgagacaagcgattttggaggaaacaagaatcccaagggcccaggaaaaccccatcttgatgcatgagttcccagataccctcttctggctttcacaggctgagctaggggttttcctgtggaaggtctgagctctccccagaaggcacttcctgtctgatgtccaggatgtcacatgttcccttacccctgtacctgtgaaggattggcagtgcttgtgcctgacctcatcaaaaaataaagctccttgtcctcattaaaaaaaa

SEQ ID NO: 2 - DNA, 500 bp, Homo sapiens
gggaccccatttctgtgctcatctgcctccttggcaatataaagagcaagcactgaggccccgcctcagagcaccccaaacttgacgccatgaagatcccggtccttcctgccgtggtgctcctctccctcctggtgctccactctgcccagggagccaccctgggtggtcctgaggaagaaagcaccattgagaattatgcgtcacgacccgaggcctttaacaccccgttcctgaacatcgacaaattgcgatctgcgtttaaggctgatgagttcctgaactggcacgccctctttgagtctatcaaaaggaaacttcctttcctcaactgggatgcctttcctaagctgaaaggactgaggagcgcaactcctgatgcccagtgaccatgacctccactggaagagggggctagcgtgagcgctgattctcaacctaccataactctttcctgcctcaggaactccaataaaacattttccatccaacaaaaaaaa

SEQ ID NO: 3 - DNA, 1366 bp, Homo sapiens
acggcttcaagctggtgatggcctccctgtaccacatctatgtggccctggaggaggagattgagcgcaacaaggagagcccagtcttcgcccctgtctacttcccagaagagctgcaccgcaaggctgccctggagcaggacctggccttctggtacgggccccgctggcaggaggtcatcccctacacaccagccatgcagcgctatgtgaagcggctccacgaggtggggcgcacagagcccgagctgctggtggcccacgcctacacccgctacctgggtgacctgtctgggggccaggtgctcaaaaagattgcccagaaagccctggacctgcccagctctggcgagggcctggccttcttcaccttccccaacattgccagtgccaccaagttcaagcagctctaccgctcccgcatgaactccctggagatgactcccgcagtcaggcagagggtgatagaagaggccaagactgcgttcctgctcaacatccagctctttgaggagttgcaggagctgctgacccatgacaccaaggaccagagcccctcacgggcaccagggcttcgccagcgggccagcaacaaagtgcaagattctgcccccgtggagactcccagagggaagcccccactcaacacccgctcccaggctccgcttctccgatgggtccttacactcagctttctggtggcgacagttgctgtagggctttatgccatgtgaatgcaggcatgctggctcccagggccatgaactttgtccggtggaaggccttctttctagagagggaattctcttggctggcttccttaccgtgggcactgaaggctttcagggcctccagccctctcactgtgtccctctctctggaaaggaggaaggagcctatggcatcttccccaacgaaaagcacatccaggcaatggcctaaacttcagagggggcgaagggatcagccctgcccttcagcatcctcagttcctgcagcagagcctggaagacaccctaatgtggcagctgtctcaaacctccaaaagccctgagtttcaagtatccttgttgacacggccatgaccactttccccgtgggccatggcaatttttacacaaacctgaaaagatgttgtgtcttgtgtttttgtcttatttttgttggagccactctgttcctggctcagcctcaaatgcagtatttttgttgtgttctgttgtttttatagcagggttggggtggtttttgagccatgcgtgggtggggagggaggtgtttaacggcactgtggccttggtctaacttttgtgtgaaataataaacaacattgtctgatagtagcttgaaaaaaaaaaaaaaaaaa

SEQ ID NO: 4 - DNA, 1490 bp, Homo sapiens
agatatccgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaa

SEQ ID NO: 5 - DNA, 1015 bp, Homo sapiens
gtgctcccggaaaggcactgggcgaccgcacccgtggctttctctgggcgaccgggtcccagactccccccagcacagcagagcgcttccctgcccacccgcggaaaccgccccaggtgggccgcgccccctccccagcagccagcagggcgccagggctgagccggccgtggaggggagcgggtcccgcgggttatacaggcgccggggctccgcggcaggatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaataaaagctcttcttttttaatatataaaagccccttaaaaaaaaaaaaaaaaaa

SEQ ID NO: 6 - DNA, 1639 bp, Homo sapiens
tcccggcctcagacacacacaccagcagctacacctacacgctgaccatcacaggcacacagaggcacatccacctcacatccacctcatacttgtgtactctcagggttcagtctttcatcctatccctctctgatctgtgcctcccaataccttccaagatgtttacagagacccttctccctgtgcagttaggagtgtaaggcaagagagcccctacttcatggggcagatcaagagctgagaccaaagatggtctatgttgctgaccttgtcctgtcctcctgctgtcttaaactatgatccctgctgcggtcactgaagcctttccctgtgagcagtggtgtgtgagagccaggcgtccctctgcctgcccactcagtggcaacacccgggagctgttttgtcctttgtggagcctcagcagttccctctttcagaactcactgccaagagccctgaacaggagccaccatgcagtgcttcagcttcattaagaccatgatgatcctcttcaatttgctcatctttctgtgtggtgcagccctgttggcagtgggcatctgggtgtcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtgtccatgtatctgtactgcaatctacaataagtccacttctgcctctgccactactgctgccacatgggaactgtgaagaggcaccctggcaagcagcagtgattgggggaggggacaggatctaacaatgtcacttgggccagaatggacctgccctttctgctccagacttggggctagatagggaccactccttttaggcgatgcctgactttccttccattggtgggtggatgggtggggggcattccagagcctctaaggtagccagttctgttgcccattcccccagtctattaaacccttgatatgccccctaggcctagtggtgatcccagtgctctactgggggatgagagaaaggcattttatagcctgggcataagtgaaatcagcagagcctctgggtggatgtgtagaaggcacttcaaaatgcataaacctgttacaatgttgccaaaaaaaaaaa

SEQ ID NO: 7 - protein, 92 aa, Homo sapiens
MSCQQNQQQCQPPPKCPSPKCPPKSPVQCLPPASSGCAPSSGGCGPSSEGGCFLNHHRRHHRCRRQRPNSCDRGSGQQGGGSGCGHGSGGCC

SEQ ID NO: 8 - protein, 99 aa, Homo sapiens
MKIPVLPAVVLLSLLVLHSAQGATLGGPEEESTIENYASRPEAFNTPFLNIDKLRSAFKADEFLNWHALFESIKRKLPFLNWDAFPKLKGLRSATPDAQ

SEQ ID NO: 9 - protein, 288 aa, Homo sapiens
MERPQPDSMPQDLSEALKEATKEVHTQAENAEFMRNFQKGQVTRDGFKLVMASLYHIYVALEEEIERNKESPVFAPVYFPEELHRKAALEQDLAFWYGPRWQEVIPYTPAMQRYVKRLHEVGRTEPELLVAHAYTRYLGDLSGGQVLKKIAQKALDLPSSGEGLAFFTFPNIASATKFKQLYRSRMNSLEMTPAVRQRVIEEAKTAFLLNIQLFEELQELLTHDTKDQSPSRAPGLRQRASNKVQDSAPVETPRGKPPLNTRSQAPLLRWVLTLSFLVATVAVGLYAM

SEQ ID NO: 10 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 11 - protein, 241 aa, Homo sapiens
MQCFSFIKTMMILFNLLIFLCGAALLAVGIWVSIDGASFLKIFGPLSSSAMQFVNVGYFLIAAGVVVFALGFLGCYGAKTESKCALVTFFFILLLIFIAEVAAAVVALVYTTMAEHFLTLLVVPAIKKDYGSQEDFTQVWNTTMKGLKCCGFTNYTDFEDSPYFKENSAFPPFCCNDNVTNTANETCTKQKAHDQKVEGCFNQLLYDIRTNAVTVGGVAAGIGGLELAAMIVSMYLYCNLQ

SEQ ID NO: 12 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 13 - DNA, 23 bp, Artificial sequence
  Synthetically generated
cgaatgtctacgtgtgtgctttg

SEQ ID NO: 14 - DNA, 20 bp, Artificial sequence
  Synthetically generated
ccggacagagcccattacaa

SEQ ID NO: 15 - DNA, 27 bp, Artificial sequence
  Synthetically generated
cgcacaaccgaagcgtagagtcacact